(19) United States
(12) Patent Application Publication
(10) Pub. No.: US 2023/0000890 A1
(43) Pub. Date: Jan. 5, 2023

(54) Prodrugs of Deoxynucleosides for Treatment of Mitochondrial Diseases Caused by Unbalanced Nucleotide Pools

(71) Applicant: ZOGENIX MDS, INC., Emeryville, CA (US)

(72) Inventor: Daniel DiPIETRO, New York, NY (US)

(21) Appl. No.: 17/841,193

(22) Filed: Jun. 15, 2022

Related U.S. Application Data
(60) Provisional application No. 63/212,468, filed on Jun. 18, 2021.

(51) Int. Cl. A61K 31/708; A61K 31/7068; A61K 9/00; A61P 43/00

(52) U.S. Cl. CPC A61K 31/708; A61K 31/7068; A61K 9/0053; A61P 43/00

(57) ABSTRACT

Deoxynucleotide prodrugs and methods of their use for treatment of diseases characterized by unbalanced nucleotide pools such as MPV17 and deoxyguanosine kinase deficiency are provided herein.

### CROSS REFERENCE TO RELATED APPLICATIONS

The present application claims priority to U.S. Provisional Application No. 63/212,468, filed Jun. 18, 2021, which is incorporated herein by reference in its entirety.

### FIELD OF THE INVENTION

The present invention relates generally to prodrugs for delivering a single nucleoside or prodrug thereof and uses of the same in treating mitochondrial DNA depletion syndrome diseases such as MPV17 and dGK deficiency disorders, optionally in combination with other nucleosides and prodrugs thereof to balance nucleotide replication pools.

### BACKGROUND OF THE INVENTION

The mitochondrion harbors its own DNA (mtDNA), which encodes many critical proteins for the assembly and activity of mitochondrial respiratory complexes (OXPHOS complexes). mtDNA is packed by many proteins to form a nucleoid that uniformly distributes within the mitochondrial matrix, which is essential for mitochondrial functions. The maintenance of mitochondrial DNA (mtDNA) depends on a number of nuclear gene-encoded proteins including a battery of enzymes forming the replisome needed to synthesize mtDNA. These enzymes need to exist in balanced quantities to function properly. In addition, mtDNA synthesis requires a balanced supply of nucleotides that is achieved by nucleotide recycling inside the mitochondria and import from the cytosol. Mitochondrial DNA maintenance defects are a group of diseases caused by pathogenic variants in the nuclear genes involved in mtDNA maintenance resulting in impaired mtDNA synthesis leading to quantitative (mtDNA depletion) and qualitative (multiple mtDNA deletions) defects in mtDNA. Defective mtDNA leads to organ dysfunction due to insufficient mtDNA-encoded protein synthesis, resulting in an inadequate energy production to meet the needs of affected organs. MDS are inherited as autosomal recessive or dominant traits and are associated with a broad phenotypic spectrum ranging from mild adult-onset ophthalmoplegia to severe infantile fatal hepatic failure. Mitochondrial diseases are clinically heterogeneous diseases due to defects of the mitochondrial respiratory chain (RC) and oxidative phosphorylation occurring in different, usually organs with intensive energy requirements and interfere with the biochemical pathways that convert energy in electrons into adenosine triphosphate (ATP).

The respiratory chain is comprised of four multi-subunit enzymes (complexes I-IV) that transfer electrons to generate a proton gradient across the inner membrane of mitochondria and the flow of protons through complex V drives ATP synthesis (DiMauro and Schon 2003; DiMauro and Hirano 2005). Coenzyme Q₁₀ (Co Q₁₀) is an essential molecule that shuttles electrons from complexes I and II to complex III. The respiratory chain is unique in eukaryotic, e.g., mammalian, cells by virtue of being controlled by two genomes, mitochondrial DNA (mtDNA) and nuclear DNA (nDNA). As a consequence, mutations in either genome can cause mitochondrial diseases. Most mitochondrial diseases affect multiple body organs and are typically fatal in the early onset forms. There are no proven effective treatments for mitochondrial diseases, only supportive therapies, such as the administration of Co Q₁₀ and its analogs to enhance respiratory chain activity and to detoxify reactive oxygen species (ROS) that are toxic by-products of dysfunctional respiratory chain enzymes. A combination of deoxycytidine and deoxythymidine is presently under study in clinical trials in several countries for treatment of TK2 deficiency disorder. (ClinicalTrials.gov Identifier: NCT03845712) The limited oral bioavailability of the purine nucleosides limits the usefulness of direct deoxyguanosine and/or deoxyadenosine supplementation for other MDSs.

Mitochondrial DNA depletion syndrome (MDS), which is a subgroup of mitochondrial disease, is a frequent cause of severe childhood encephalomyopathy, hepatocerebral or hepato-specific disease characterized molecularly by reduction of mitochondrial DNA (mtDNA) copy number or accumulation of mtDNA depletions in tissues and insufficient synthesis of mitochondrial RC complexes (Hirano, et al. 2001). Mutations in several nuclear genes have been identified as causes of infantile MDS, including: TK2, DGUOK (or dGK as used in some instances herein, which distinguishes from DGK that designates diacylglycerol kinase), POLG, POLG2, SCLA25A4, MPV17, RRM2B, SUCLA2, SUCLG1, TYMP, OPA1, and ClOorfl (PEOl). (Bourdon, et al. 2007; Copeland 2008; Elpeleg, et al. 2005; Mandel, et al. 2001; Naviaux and Nguyen 2004; Ostergaard, et al. 2007; Saada, et al. 2003; Sarzi, et al. 2007; Spinazzola, et al, 2006). In addition, mutations in these nuclear genes can also cause multiple deletions of mtDNA with or without mtDNA depletion (Behin, et al. 2012; Garone, et al. 2012; Longley, et al. 2006; Nishino, et al. 1999; Paradas, et al. 2012; Ronchi, et al. 2012; Spelbrink, et al. 2001; Tyynismaa, et al. 2009; Tyynismaa, et al. 2012; Van Goethem, et al. 2001).

Deoxyguanosine kinase (dGK: dG (deoxyguanosine) kinase) is an essential rate-limiting component of the mitochondrial purine nucleotide salvage pathway, encoded by the nuclear gene (DGUOK) encoding deoxyguanosine kinase. Mutations in DGUOK lead to mtDNA depletion typically in the liver and brain, causing a hepatocerebral phenotype and it is one of the two mitochondrial deoxynucleoside salvage pathway enzymes involved in precursor synthesis for mtDNA replication. dGK is responsible for the initial rate-limiting phosphorylation of the purine deoxynucleosides, using a nucleoside triphosphate as phosphate donor. Mutations in the DGOUK gene are associated with the hepato-specific and hepatocerebral forms of MDS (mtDNA depletion syndrome) diseases. The deoxy monophosphates are in turn converted to di-and triphosphates for incorporation into mtDNA. Previously it was shown in cultured cells that supplementation with 50 μm deoxyguanosine alone added into culture broth was sufficient to increase the mtDNA copy number in fibroblasts from patients with DGUOK mutations (Camara, et al. 2014). More recently, a zebrafish mutant dGK model was generated and these homozygous mutants exhibit the characteristic decrease in mtDNA amounts, but did not have a visible phenotype. A possible explanation for this could be compensation by the cytoplasmic enzyme deoxycytidine kinase (dCK). The attempt to increase the mtDNA levels in the dGK mutant fish by adding deoxyguanosine alone, similar to the experiments performed in fibroblast cell culture, resulted in decreased mtDNA levels in the model. It was suggested that the concentration of just one of the substrate nucleosides may introduce an imbalance in the dNTP pool and thereby interferes with mtDNA replication and as a consequence reduces the mtDNA copy number. However in a further experiment that supplemented the dGK mutant fish with both purine nucleosides a significant increase in liver mtDNA copy number was detected (Munro, et al. 2019).

### dGK Clinical Presentation

Disease on-set is predominantly in the neonatal period, infancy, or childhood. It is an autosomal recessive inheritance disorder resulting in mitochondrial depletion. The disease manifests with symptoms of hepatopathy and encephalopathy. Patients usually initially present with hypoglycemia and lactic acidosis. Additionally, elevated serum concentrations of tyrosine or phenylalanine are evident on a newborn screen in affected neonates, and liver transaminases, gamma glutamyl transferase (GGT), and conjugated hyperbilirubinemia are typically elevated. Patients have a variable neuromuscular phenotype, but symptoms may include developmental regression, hypotonia, severe myopathy, rotary nystagmus, and opsoclonus.

Mpv17 was identified through disease-segregating mutations in three families with hepatocerebral MDS and demonstrated that MPV17 is a mitochondrial inner membrane protein, and its absence or malfunction causes oxidative phosphorylation (OXPHOS) failure and mtDNA depletion, not only in affected individuals but also in Mpv17 −/− mice. (Spinazzola, et al. 2006). More recent reports have shown that MPV17 is protein involved in importing deoxynucleotides into the mitochondria and its dysfunction causes disease in both its infantile-onset and, more rarely, in adult-onset forms. The infantile-onset form is characterized as a severe hepato-cerebral MDS and the adult-onset form by a neuromuscular or multisystemic phenotype with deletions. (El-hattab, et al. 2018).

There is a need for treatments for many forms of MDS and other diseases characterized by unbalanced nucleotide pools as has been described, for example, in U.S. Pat. No. 10,471,087. For example, several mendelian disorders with mtDNA depletion or multiple deletions, or both are characterized by unbalanced deoxynucleotide triphosphate pools that lead to defects of mtDNA replication. Other nuclear genes that disrupt mitochondrial dNTP pools include TK2, TYMP, RRM2B, SUCLA2, SUCLG1 and POLG. Therapies that restore dNTP pool balance would be useful to treat these disorders as well, especially administration of a single pro-drug agent would provide dosing and patient compliance advantages.

### SUMMARY OF THE INVENTION

The present invention relates generally to prodrugs of deoxyguanosine for delivering the deoxynucleosides or deoxynucleotides to affected tissues in dGK or MPV17 deficiency disorders, optionally including one or more prodrugs of other nucleosides/nucleotides intended to balance nucleotide pools. It has been surprisingly found that treatment with (dG) deoxyguanosine alone, or dG administered as a prodrug according to the present invention alone, is sufficient to increase mtDNA copy numbers in dGK or MPV17 deficiency disorders.

In one aspect, the present invention provides compounds of Formula I for nucleoside supplementation treatments for mitochondrial depletion syndromes:

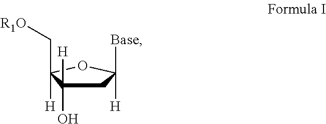

wherein Base refers to an optionally substituted heterocyclic base or an optionally substituted heterocyclic base with a protected amino group;

R¹ is selected from the group consisting of optionally substituted acyl, optionally substituted O-linked amino acid,

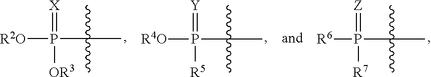

X, Y and Z are each independently selected from O and S;

R², R³ and R⁴ are each independently selected from hydrogen, optionally substituted C₁₋₂₄ alkyl, optionally substituted C₂₋₂₄ alkenyl, optionally substituted C₂₋₂₄ alkynyl, optionally substituted C₃₋₆ cycloalkyl, optionally substituted C₃₋₆ cycloalkenyl, optionally substituted aryl, optionally substituted heteroaryl, optionally substituted aryl(C₁₋₆)alkyl,

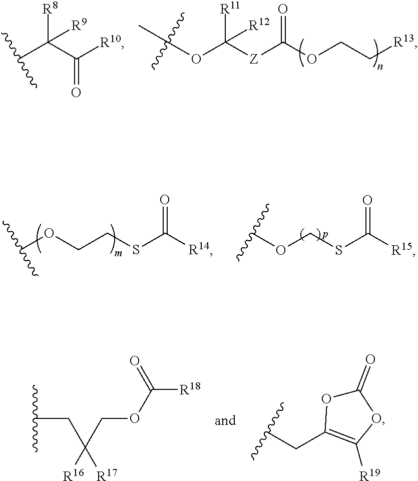

or R² and R³ can be taken together to form a cyclic moiety;

R⁵, R⁶ and R⁷ are each independently selected from optionally substituted C₁₋₂₄ alkyl, optionally substituted C₂₋₂₄ alkenyl, optionally substituted C₂₋₂₄ alkynyl, optionally substituted C₃₋₆ cycloalkyl, optionally substituted C₃₋₆ cycloalkenyl, NR²⁰R²¹, optionally substituted N-linked amino acid, optionally substituted N-linked amino acid ester;

R⁸, R⁹, R¹¹ and R¹² are each independently selected from hydrogen, optionally substituted C1-24 alkyl and optionally substituted aryl;

R¹⁰ and R¹³ are each independently selected from hydrogen, optionally substituted C1-24 alkyl and optionally substituted aryl, an optionally substituted —O—C1-24 alkyl, an optionally substituted —O-aryl, an optionally substituted —O-heteroaryl, an optionally substituted —O-monocyclic hetercyclyl;

R¹⁴, R¹⁵ and R¹⁹ are each independently selected from hydrogen, an optionally substituted C₁₋₂₄ alkyl and an optionally substituted aryl;

R¹⁶ and R¹⁷ are each independently selected from —CN, optionally substituted C₂₋₈ organylcarbonyl, C₂₋₈ alkoxycarbonyl and C₂₋₈ organylaminocarbonyl;

R¹⁸ is selected from hydrogen, optionally substituted C₁₋₂₄ alkyl, optionally substituted C₂₋₂₄ alkenyl, optionally substituted C₂₋₂₄ alkynyl; optionally substituted C₃₋₆ cycloalkyl and optionally substituted C₃₋₆ cycloakenyl;

R²⁰ and R²¹ are each independently selected from hydrogen, optionally substituted C₁₋₂₄ alkyl, optionally substituted C₂₋₂₄ alkenyl, optionally substituted C₂₋₂₄ alkynyl; optionally substituted C₃₋₆ cycloalkyl and optionally substituted C₃₋₆ cycloakenyl; and

n, m and p are each independently selected from 0, 1, 2, or 3.

In another aspect, the prodrugs are nucleoside/mononucleotide compounds of Formula II:

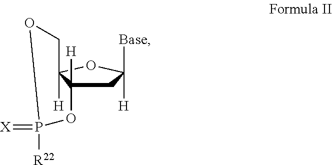

wherein Base refers to an optionally substituted heterocyclic base or an optionally substituted heterocyclic base with a protected amino group;

X is selected from S and O; and

R²² is selected from —O⁻, —OH, —O-alkyl, optionally substituted C₁₋₆ alkoxy,

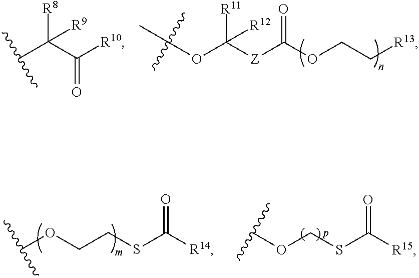

optionally substituted N-linked amino acid and optionally substituted N-linked amino acid ester;

wherein R⁸, R⁹, R¹⁰, R¹¹, R¹², R¹³, R¹⁴, R¹⁵, n, m and p are defined as above.

In still another aspect, the prodrugs are compounds of Formula III:

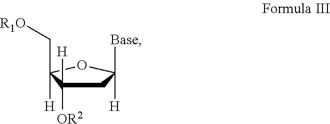

wherein Base refers to an optionally substituted heterocyclic base or an optionally substituted heterocyclic base with a protected amino group;

R¹ and R² are independent selected from hydrogen, phosphate (including mono-, di-, or tri-phosphate and the modified phosphates of Formula I); straight chained, branched or cyclic alkyl; acyl; CO-alkyl, CO-alkoxyalkyl; CO-aryloxyalkyl, CO-substituted aryl, sulfonate ester; alkylsulfonyl; arylsulfonyl; aralkylsulfonyl; a lipid; a phospholipid; an amino acid; a carbohydrate; a peptide and cholesterol.

In one embodiment, Base in each of Formula I, II and III refers to guanine.

In one embodiment, Base in each of Formula I, II and III refers to guanine with an amine protecting group. Examples of amino protecting groups include without limitation, carbamate-protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyl-oxycarbonyl (Cbz); amide-protecting groups, such as formyl, acetyl, trihaloacetyl, benzoyl, and nitrophenylacetyl; sulfonamide-protecting groups, such as 2-nitrobenzenesulfonyl; and imine- and cyclic imide-protecting groups, such as phthalimido and dithiasuccinoyl.

In yet another aspect, the present invention also generally relates to methods of treating a disease or disorder characterized by unbalanced nucleotide pools in a subject in need thereof comprising administering to the subject a therapeutically effective amount of at least one prodrug of the present invention. The prodrug can be administered as such (i.e. alone) or in the form of a pharmaceutical composition.

Suitable diseases or disorders include, but are not limited to, MPV17 deficiency and dGK mutations.

Administration can be via any route including, but not limited to, oral, gastric feeding tube, intrathecal, parental, mucosal and transdermal administration.

The amount of the at least one prodrug or composition comprising the same administered can be from about 15 mg/kg/day to about 500 mg/kg/day.

### BRIEF DESCRIPTION OF THE DRAWINGS

FIGS. 1A, 1B, and 1C compare levels of nucleosides in MPV17 knockout mice and normal controls in liver, kidney and brain, respectively. FIG. 1A displays the marked depletion of dTTP and dGTP in liver mitochondria. The results further show that liver mitochondria (FIG. 1A) in MPV 17^{-/-} mice reveal shortages of dGTP and dTTP deoxynucleotides, but not kidney mitochondria (FIG. 1B) or brain mitochondria (FIG. 1C).

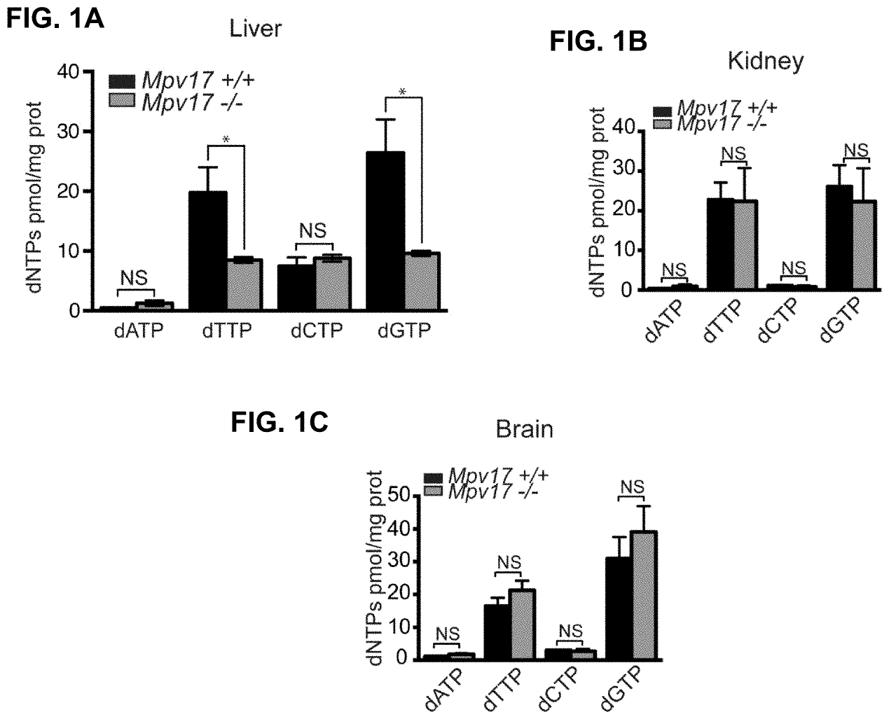

FIG. 2 displays results of an earlier study with nucleoside supplementation in MPV17 deficient human fibroblasts. FIG. 2 shows various combinations of nucleosides added to the cell culture broth. The abbreviation GdR corresponds to dG, TdR to dT and A/CdR corresponds to a mixture of dA and dC and so on.

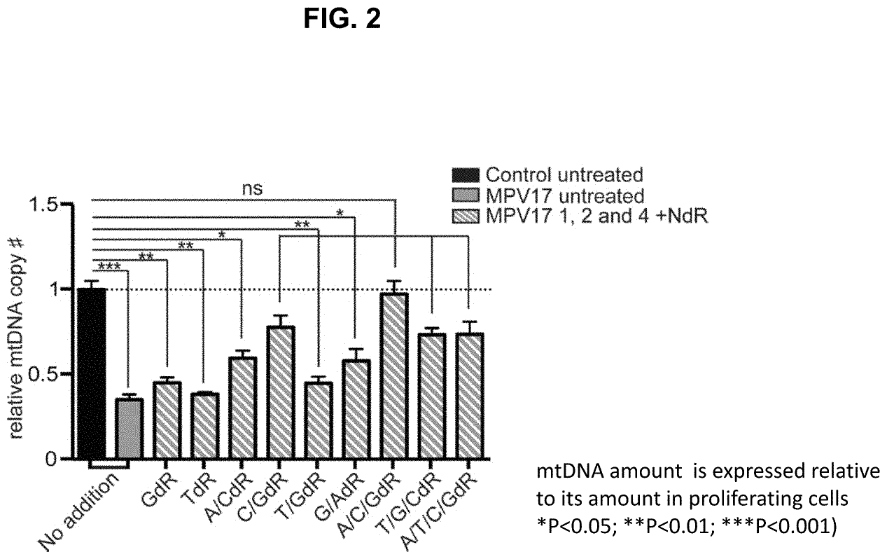

FIG. 3 presents a synthesis scheme for 3′Val-dG (MT101G) and is further described in the Examples below.

FIG. 4 presents a synthesis scheme for 3′Val-dA (MT101A) and is further described in the Examples below.

FIG. 5 presents a synthesis scheme for 3′Val, 5′isobutryl dG (MT104G) and is further described in the Examples below.

FIG. 6A presents a starting material, yield and structure for 3′,5′diisobutryl dG (MT104G). FIG. 6B presents a starting material, yield and structure for 3′,5′diisobutryl dA (MT104A). Both of which are further described in the Examples below.

FIG. 7 presents a bar graph of the results of qPCR measurements of mtDNA copy number in the liver MPV17^{-/-} mice in treated and untreated groups. The graph represents Mean±SEM for each experimental group. All the treatments represent a molar equivalent of 75 mg/kg dC or 75 mg/kg dG.

FIGS. 8A, 8B, and 8C present results of further studies on the effects of prodrug treatments of MPV17^{-/-} mice. FIGS. 8A, 8B, and 8C show that body weight increases similarly between experimental and untreated groups. FIG. 8A presents the combinations of cohorts of knock out (KO) litters and dosing regimens. FIGS. 8B and 8C show plots of the body weight increases of pups in treated and untreated groups.

FIG. 8D presents a bar graph of the results of qPCR measurements of mtDNA copy number in the liver of MPV17^{-/-} mice per dosing group, comparing wild type, untreated MPV17^{-/-} mice, and treated MPV17^{-/-} mice treated with MT101 and MT104 comprising combinations of dG and dC. The graph represents Mean±SD for each experimental group. Values for each experimental group were compared to those obtained in untreated MPV17^{-/-} animals with Kruskal-Wallis test followed by Dunn's non-parametric test. P values are *p<0.05 and ***p>0.001. 20% and 50% represent the molar equivalent to 75 to 200 mg/kg dG, respectively.

FIGS. 8E, 8F, and 8G present bar graphs of several OXPHOS (oxidative phosphorylation) component protein levels comparing wild type, untreated MPV17^{-/-} knockout mice, and treated MPV17^{-/-} mice treated with MT101 and MT104 of the invention comprising combinations of dG and dC. OXPHOS components of complex I (Ndufb8), complex III (Core2), and complex V (Atp5a) were determined for each dosing regimen as shown in FIG. 8D.

FIG. 9 presents a bar graph of the results of qPCR measurements of mtDNA copy number in the liver MPV17^{-/-} mice. FIG. 9 compares wild type, untreated MPV17^{-/-} mice, and treated MPV17^{-/-} mice with a single nucleoside prodrug and underivatized dG and dC. The graph represents Mean±SEM for each experimental group. All treatments represent molar equivalents of 75 mg/kg dC and/or 75 mg/kg dG.

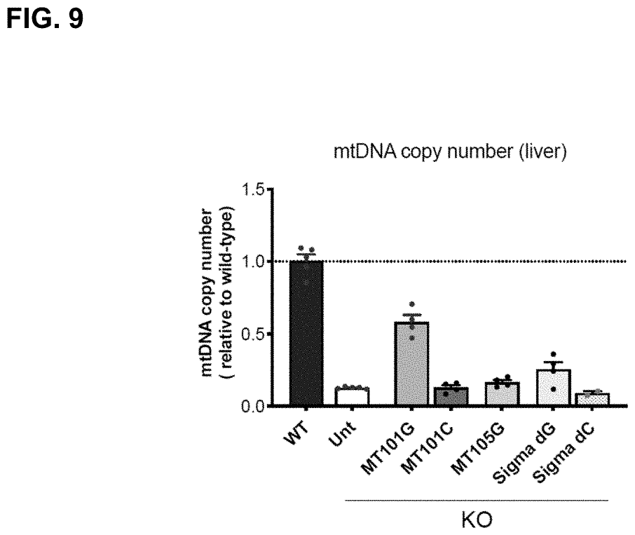

### DETAILED DESCRIPTION OF THE INVENTION

### I. Definitions

As used herein, “subject” refers to mammals. Mammals include canines, felines, rodents, bovine, equines, porcines, ovines, and primates. Thus, the invention can be used in veterinary medicine, e.g., to treat companion animals, farm animals, laboratory animals in zoological parks, and animals in the wild. The invention is particularly desirable for human medical applications

As used herein, “patient” refers to a human subject. In some embodiments of the present invention, the “patient” is known or suspected of having a disease or disorder characterized by unbalanced nucleotide pools, mitochondrial disease, mitochondrial DNA depletion syndrome, or dGK or MPV17 deficiency disease.

As used herein, “therapeutically effective amount” refers to an amount sufficient to cause an improvement in a clinically significant condition in the subject, or delays or minimizes or mitigates one or more symptoms associated with the disease or disorder, or results in a desired beneficial change of physiology in the subject.

As used herein, “treat”, “treatment”, and the like refer to a means to slow down, relieve, ameliorate or alleviate at least one of the symptoms of the disease or disorder, or reverse the disease or disorder after its onset.

As used herein, “prevent”, “prevention”, and the like refer to acting prior to overt disease or disorder onset, to prevent the disease or disorder from developing or minimize the extent of the disease or disorder, or slow its course of development.

As used herein, “in need thereof” refers to a subject known or suspected of having or being at risk of having a disease or disorder characterized by unbalanced nucleotide pools, mitochondrial disease, mitochondrial DNA depletion syndrome, such as MPV17 deficiency or dGK deficiency diseases.

As used herein, “prodrug” refers to a derivative of the deoxynucleoside that undergoes a transformation under the conditions of use, such as within the body, to release the deoxynucleoside. Prodrugs are frequently, but not necessarily, pharmacologically inactive until converted into the active form. Prodrugs can be obtained by bonding a promoiety, typically via a functional group, to a drug.

As used herein, “promoiety” refers to a group bonded to the deoxynucleoside, typically to a functional group, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. The cleavage of the promoiety may proceed spontaneously, such as via a hydrolysis reaction, or it may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation of a patient to which the prodrug is administered or the acidic conditions of the stomach or the agent may be supplied exogenously.

As used herein, “an adverse effect” is an unwanted reaction caused by the administration of a drug. In most cases, the administration of the deoxynucleosides caused no adverse effects. The most expected adverse effect would be a minor gastrointestinal intolerance.

As used herein, “about” or “approximately” means within an acceptable error range for the particular value as determined by one of ordinary skill in the art, which will depend in part on how the value is measured or determined, i.e. , the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

Whenever a group is described as “optionally substituted” that group may be unsubstituted or substituted with one or more of the indicated substituents. Likewise, when a group is described as “unsubstituted or substituted” if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, aryl, heteroaryl, heterocyclyl, aryl(alkyl), heteroaryl(alkyl), heterocyclyl(alkyl), hydroxy, alkoxy, aryloxy, acyl, mercapto, alkylthio, arylthio, cyano, halogen, thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, azido, nitro, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group, and protected derivatives thereof.

The term “protecting group,” or “protected derivative” as used herein, refers to a labile chemical moiety which is known in the art to protect reactive groups including without limitation, hydroxyl, amino and thiol groups, against undesired reactions, typically during synthetic procedures. Protecting groups are typically used selectively and/or orthogonally to protect sites during reactions at other reactive sites and can then be removed to leave the unprotected group as is or available for further reactions. Protecting groups as known in the art are described generally in Greene's Protective Groups in Organic Synthesis, 4th edition, John Wiley & Sons, New York, 2007.

Groups can be selectively incorporated into compounds as provided herein as precursors. For example, an amino group can be placed into a compound as provided herein as an azido group that can be chemically converted to the amino group at a desired point in the synthesis. Generally, groups are protected or present as precursors that will be inert to reactions that modify other areas of the parent molecule for conversion into their final groups at an appropriate time. Further representative protecting or precursor groups are discussed in Agrawal et al., Protocols for Oligonucleotide Conjugates, Humana Press; New Jersey, 1994, 26, 1-72. Alternatively, the protecting group can remain as a constituent of the final product.

Examples of amino protecting groups include without limitation, carbamate-protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyl-oxycarbonyl (Cbz); amide-protecting groups, such as formyl, acetyl, trihaloacetyl, benzoyl, and nitrophenylacetyl; sulfonamide-protecting groups, such as 2-nitrobenzenesulfonyl; and imine- and cyclic imide-protecting groups, such as phthalimido and dithiasuccinoyl.

Examples of hydroxyl protecting groups include without limitation, acetyl, t-butyl, t-butoxymethyl, methoxymethyl, tetrahydropyranyl, 1-ethoxyethyl, 1-(2-chloroethoxy)ethyl, p-chlorophenyl, 2,4-dinitrophenyl, benzyl, 2,6-dichlorobenzyl, diphenylmethyl, p-nitrobenzyl, bis(2-acetoxyethoxy)methyl (ACE), 2-trimethylsilylethyl, trimethylsilyl, triethylsilyl, t-butyldimethylsilyl, t-butyldiphenylsilyl, triphenylsilyl, [(triisopropylsilyl)oxy]methyl (TOM), benzoylformate, chloroacetyl, trichloroacetyl, trifluoro-acetyl, pivaloyl, benzoyl, p-phenylbenzoyl, 9-fluorenylmethyl carbonate, mesylate, tosylate, triphenylmethyl (trityl), monomethoxytrityl, dimethoxytrityl (DMT), trimethoxytrityl, 1(2-fluorophenyl)-4-methoxypiperidin-4-yl (FPMP), 9-phenylxanthine-9-yl (Pixyl) and 9-(p-methoxyphenyl)xanthine-9-yl (MOX). Wherein more commonly used hydroxyl protecting groups include without limitation, benzyl, 2,6-dichlorobenzyl, t-butyldimethylsilyl, t-butyldiphenylsilyl, benzoyl, mesylate, tosylate, dimethoxytrityl (DMT), 9-phenylxanthine-9-yl (Pixyl) and 9-(p-methoxyphenyl)xanthine-9-yl (MOX).

Examples of protecting groups commonly used to protect phosphate and phosphorus hydroxyl groups include without limitation, methyl, ethyl, benzyl (Bn), phenyl, isopropyl, tent-butyl, allyl, cyclohexyl (cHex), 4-methoxybenzyl, 4-chlorobenzyl, 4-nitrobenzyl, 4-acyloxybenzyl, 2-methylphenyl, 2,6-dimethylphenyl, 2-chlorophenyl, diphenylmethyl, 4-methylthio-1-butyl, 2-(S-Acetylthio)ethyl (SATE), 2-cyanoethyl, 2-cyano-1,1-dimethylethyl (CDM), 4-cyano-2-butenyl, 2-(trimethylsilyl)ethyl (TSE), 2-(phenylthio)ethyl, 2-(triphenylsilyl)ethyl, 2-(benzylsulfonyl)ethyl, 2,2,2-trichloroethyl, 2,2,2-tribromoethyl, 2,3-dibromopropyl, 2,2,2-trifluoroethyl, thiophenyl, 2-chloro-4-tritylphenyl, 2-bromophenyl, 2-[N-isopropyl-N-(4-methoxybenzoyl)amino]ethyl, 4-(N-trifluoroacetylamino)butyl, 4-oxopentyl, 4-tritylaminophenyl, 4-benzylaminophenyl and morpholino. Wherein more commonly used phosphate and phosphorus protecting groups include without limitation, methyl, ethyl, benzyl (Bn), phenyl, isopropyl, tert-butyl, 4-methoxybenzyl, 4-chlorobenzyl, 2-chlorophenyl and 2-cyanoethyl.

As used herein, “Ca to Cb” in which “a” and “b” are integers refer to the number of carbon atoms in an alkyl, alkenyl or alkynyl group, or the number of carbon atoms in the ring of a cycloalkyl, cycloalkenyl, aryl, heteroaryl or heterocyclyl group. That is, the alkyl, alkenyl, alkynyl, ring(s) of the cycloalkyl, ring(s) of the cycloalkenyl, ring(s) of the aryl, ring(s) of the heteroaryl or ring(s) of the heterocyclyl can contain from “a” to “b”, inclusive, carbon atoms. Thus, for example, a “C1 to C4 alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, alkenyl, alkynyl, cycloalkyl cycloalkenyl, aryl, heteroaryl or heterocyclyl group, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 10 carbon atoms. The alkyl group could also be a lower alkyl having 1 to 6 carbon atoms. The alkyl group of the compounds may be designated as “C1-C4 alkyl” or similar designations. By way of example only, “C1-C4 alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

As used herein, “alkenyl” refers to an alkyl group that contains in the straight or branched hydrocarbon chain one or more double bonds. Examples of alkenyl groups include allenyl, vinylmethyl and ethenyl. An alkenyl group may be unsubstituted or substituted.

As used herein, “alkynyl” refers to an alkyl group that contains in the straight or branched hydrocarbon chain one or more triple bonds. Examples of alkynyls include ethynyl and propynyl. An alkynyl group may be unsubstituted or substituted.

As used herein, “cycloalkyl” refers to a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

As used herein, “cycloalkenyl” refers to a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl can contain 3 to 10 atoms in the ring(s) or 3 to 8 atoms in the ring(s). A cycloalkenyl group may be unsubstituted or substituted.

As used herein, “aryl” refers to a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C6-C14 aryl group, a C6-C10 aryl group, or a C6 aryl group. Examples of aryl groups include, but are not limited to, benzene, naphthalene and azulene. An aryl group may be substituted or unsubstituted.

As used herein, “heteroaryl” refers to a monocyclic, bicyclic and tricyclic aromatic ring system (a ring system with fully delocalized pi-electron system) that contain(s) one or more heteroatoms (for example, 1 to 5 heteroatoms), that is, an element other than carbon, including but not limited to, nitrogen, oxygen and sulfur. The number of atoms in the ring(s) of a heteroaryl group can vary. For example, the heteroaryl group can contain 4 to 14 atoms in the ring(s), 5 to 10 atoms in the ring(s) or 5 to 6 atoms in the ring(s). Furthermore, the term “heteroaryl” includes fused ring systems where two rings, such as at least one aryl ring and at least one heteroaryl ring, or at least two heteroaryl rings, share at least one chemical bond. Examples of heteroaryl rings include, but are not limited to, furan, furazan, thiophene, benzothiophene, phthalazine, pyrrole, oxazole, benzoxazole, 1,2,3-oxadiazole, 1,2,4-oxadiazole, thiazole, 1,2,3-thiadiazole, 1,2,4-thiadiazole, benzothiazole, imidazole, benzimidazole, indole, indazole, pyrazole, benzopyrazole, isoxazole, benzoisoxazole, isothiazole, triazole, benzotriazole, thiadiazole, tetrazole, pyridine, pyridazine, pyrimidine, pyrazine, purine, pteridine, quinoline, isoquinoline, quinazoline, quinoxaline, cinnoline and triazine. A heteroaryl group may be substituted or unsubstituted.

As used herein, “heterocyclyl” or “heteroalicyclyl” refers to three-, four-, five-, six-, seven-, eight-, nine-, ten-, up to 18-membered monocyclic, bicyclic, and tricyclic ring system wherein carbon atoms together with from 1 to 5 heteroatoms constitute said ring system. A heterocycle may optionally contain one or more unsaturated bonds situated in such a way, however, that a fully delocalized pi-electron system does not occur throughout all the rings. The heteroatom(s) is an element other than carbon including, but not limited to, oxygen, sulfur, and nitrogen. A heterocycle may further contain one or more carbonyl or thiocarbonyl functionalities, so as to make the definition include oxo-systems and thio-systems such as lactams, lactones, cyclic imides, cyclic thioimides and cyclic carbamates. When composed of two or more rings, the rings may be joined together in a fused fashion. Additionally, any nitrogens in a heteroalicyclic may be quaternized. Heterocyclyl or heteroalicyclic groups may be unsubstituted or substituted. Examples of such “heterocyclyl” or “heteroalicyclyl” groups include but are not limited to, 1,3-dioxin, 1,3-dioxane, 1,4-dioxane, 1,2-dioxolane, 1,3-dioxolane, 1,4-dioxolane, 1,3-oxathiane, 1,4-oxathiin, 1,3-oxathiolane, 1,3-dithiole, 1,3-dithiolane, 1,4-oxathiane, tetrahydro-1,4-thiazine, 2H-1,2-oxazine, maleimide, succinimide, barbituric acid, thiobarbituric acid, dioxopiperazine, hydantoin, dihydrouracil, trioxane, hexahydro-1,3,5-triazine, imidazoline, imidazolidine, isoxazoline, isoxazolidine, oxazoline, oxazolidine, oxazolidinone, thiazoline, thiazolidine, morpholine, oxirane, piperidine N-Oxide, piperidine, piperazine, pyrrolidine, pyrrolidone, pyrrolidione, 4-piperidone, pyrazoline, pyrazolidine, 2-oxopyrrolidine, tetrahydropyran, 4H-pyran, tetrahydrothiopyran, thiamorpholine, thiamorpholine sulfoxide, thiamorpholine sulfone, and their benzo-fused analogs (e.g., benzimidazolidinone, tetrahydroquinoline, and 3,4-methylenedioxyphenyl).

As used herein, “aralkyl” and “aryl(alkyl)” refer to an aryl group connected, as a substituent, via a lower alkylene group. The lower alkylene and aryl group of an aryl(alkyl) may be substituted or unsubstituted. Examples include but are not limited to benzyl, 2-phenyl(alkyl), 3-phenyl(alkyl), and naphthyl(alkyl).

As used herein, “heteroaralkyl” and “heteroaryl(alkyl)” refer to a heteroaryl group connected, as a substituent, via a lower alkylene group. The lower alkylene and heteroaryl group of heteroaryl(alkyl) may be substituted or unsubstituted. Examples include but are not limited to 2-thienyl(alkyl), 3-thienyl(alkyl), furyl(alkyl), thienyl(alkyl), pyrrolyl(alkyl), pyridyl(alkyl), isoxazolyl(alkyl), imidazolyl(alkyl), and their benzo-fused analogs.

As used herein, “alkoxy” refers to the formula —OR wherein R is an alkyl, an alkenyl, an alkynyl, a cycloalkyl, a cycloalkenyl, aryl, heteroaryl, heteroalicyclyl, aralkyl, heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen an alkyl, an alkenyl, an alkynyl, a cycloalkyl, a cycloalkenyl, aryl, heteroaryl, heteroalicyclyl, aralkyl, heteroaryl(alkyl) or heterocyclyl(alkyl) connected, as substituents, via a carbonyl group. Examples include formyl, acetyl, propanoyl, benzoyl, and acryl. An acyl may be substituted or unsubstituted.

As used herein, the term “heterocyclic base” refers to an optionally substituted nitrogen-containing heterocyclyl that can be attached to an optionally substituted pentose moiety or modified pentose moiety. In some embodiments, the heterocyclic base can be selected from an optionally substituted purine-base, an optionally substituted pyrimidine-base and an optionally substituted triazole-base (for example, a 1,2,4-triazole). The term “purine-base” is used herein in its ordinary sense as understood by those skilled in the art and includes its tautomers. Similarly, the term “pyrimidine-base” is used herein in its ordinary sense as understood by those skilled in the art and includes its tautomers. A non-limiting list of optionally substituted purine-bases includes purine, adenine, guanine, hypoxanthine, xanthine, alloxanthine, 7-alkylguanine (e.g. 7-methylguanine), theobromine, caffeine, uric acid and isoguanine. Preferred purine bases are adenine and guanine including amine and/or enol protected adenine and guanine bases. Examples of pyrimidine-bases include, but are not limited to, cytosine, thymine, uracil, 5,6-dihydrouracil and 5-alkylcytosine (e.g., 5-methylcytosine). Preferred pyrimidine bases are cytosine and thymidine including amine or enol protected cytosine and thymidine bases. An example of an optionally substituted triazole-base is 1,2,4-triazole-3-carboxamide. Other non-limiting examples of heterocyclic bases include diaminopurine, 8-oxo-N⁶-alkyladenine (e.g., 8-oxo-N⁶-methyladenine), 7-deazaxanthine, 7-deazaguanine, 7-deazaadenine, N⁴,N⁴-ethanocytosin, N⁶,N⁶-ethano-2,6-diaminopurine, 5-halouracil (e.g., 5-fluorouracil and 5-bromouracil), pseudoisocytosine, isocytosine, isoguanine, and other heterocyclic bases described in U.S. Pat. Nos. 5,432,272 and 7,125,855, which are incorporated herein by reference for the limited purpose of disclosing additional heterocyclic bases. In some embodiments, a heterocyclic base can be optionally substituted with an amine or an enol protecting group.

As used herein, “—N-linked amino acid” refers to an amino acid that is attached to the indicated moiety via a main-chain amino or mono-substituted amino group. When the amino acid is attached in an —N-linked amino acid, one of the hydrogens that is part of the main-chain amino or mono-substituted amino group is not present and the amino acid is attached via the nitrogen. N-linked amino acids can be substituted or unsubstituted.

As used herein, “—N-linked amino acid ester” refers to an amino acid in which a main-chain carboxylic acid group has been converted to an ester group. In some embodiments, the ester group has a formula selected from alkyl-O—C(═O)—, cycloalkyl-O—C(═O)—, aryl-O—C(═O)— and aryl(alkyl)-O—C(═O)—. A non-limiting list of ester groups include substituted and unsubstituted versions of the following: methyl-O—C(═O)—, ethyl-O—C(═O)—, n-propyl-O—C(═O)—, isopropyl-O—C(═O)—, n-butyl-O—C(═O)—, isobutyl-O—C(═O)—, tert-butyl-O—C(═O)—, neopentyl-O—C(═O)—, cyclopropyl-O—C(═O)—, cyclobutyl-O—C(═O)—, cyclopentyl-O—C(═O)—, cyclohexyl-O—C(═O)—, phenyl-O—C(═O)—, benzyl-O—C(═O)—, and naphthyl-O—C(═O)—. N-linked amino acid ester derivatives can be substituted or unsubstituted.

As used herein, “—O-linked amino acid” refers to an amino acid that is attached to the indicated moiety via the hydroxy from its main-chain carboxylic acid group. When the amino acid is attached in an —O-linked amino acid, the hydrogen that is part of the hydroxy from its main-chain carboxylic acid group is not present and the amino acid is attached via the oxygen. O-linked amino acids can be substituted or unsubstituted.

As used herein, the term “amino acid” refers to any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, gamma-aminobutyric acid, citrulline, beta-alanine, alpha-ethyl-glycine, alpha-propyl-glycine and norleucine.

The term “amino acid” includes naturally occuning and synthetic, β γ or δ amino acids, The amino acid can be in the D- or L-configuration. The amino acid can be a derivative of alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-isoleuccinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl.

The term “deoxynucleoside”, as used herein, refers to any nucleoside containing a deoxy sugar, i.e., any compound formally derived from a sugar by replacing a hydroxy group by a hydrogen atom, e.g., deoxyribose.

The term “deoxynucleoside prodrug” or “prodrug deoxynucleoside” as used herein, refers to prodrug derivatives of a deoxynucleoside and includes prodrugs that incorporate a phosphate group, or a derivative thereof, into their structure.

The term “dNTP”, as used herein, refers to deoxyribonucleotide triphosphate. Each dNTP is made up of a phosphate group, a deoxyribose sugar and a nitrogenous base. There are four different dNTPs and can be split into two groups: the purines and the pyrimidines. dATP, (deoxyadenosine 5′-triphosphate), and dGTP, (deoxyguanine 5′-triphosphate), make up the purines, while dTTP, (deoxythymidine 5′-triphosphate), and dCTP, (deoxycytidine 5′-triphosphate), make up the pyrimidines. Adenine and guanine, the bases which feature in the purines, both have a double ring structure, while thymine and cytosine, the bases which feature in the purines, both have a single ring structure.

The term “mitochondrial DNA depletion syndrome”, as used herein, refers to a class of phenotypically diverse diseases and disorders characterized by a severe reduction in mitochondrial DNA (mtDNA) content in affected tissues and organs, for example, muscle, liver brain and/or GI tract. The reduction or depletion can result from any imbalance in the mitochondrial nucleotide pool available for mtDNA replication, as well as abnormalities in mitochondrial replication. Based on age at onset, two subtypes are distinguished: congenital (or early-onset) and infantile (or later-onset). Although survival is longer in the later-onset form, the syndrome is fatal in almost all patients, and currently there is no effective treatment.

### II. Prodrugs

The present invention provides deoxynucleoside prodrugs for use in the treatment of MDS. “Deoxynucleoside” refers to 2′-deoxynucleosides, e.g. 2′-deoxyguanosine (dG, or deoxyguanosine, shown below), 2′-deoxythymidine (dT or thymdine), 2′-deoxyadenosine (dA or deoxyadenosine) and 2′-deoxycytidine (dC or deoxycytidine). The full-length names and common abbreviation for each can be used interchangeably:

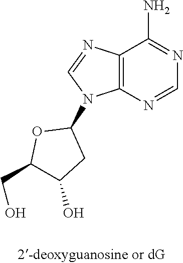 [SMILES: Nc1ncnc2c1ncn2[C@H]1C[C@H](O)[C@@H](CO)O1]

In particular embodiments, Base is selected from cytosine, thymine, guanine and adenine.

As such, the prodrugs describe herein are deoxycytidine prodrugs (dC prodrugs), deoxythymidine prodrugs (dT prodrugs), deoxyguanosine prodrugs (dG prodrugs) and deoxyadenosine prodrugs (dA prodrugs).

The prodrugs are preferably in the naturally-occurring β-D-configuration of the Base and deoxyribose moieties and the connection point of the pyrimidine and purine rings to Formula I is preferable the naturally-occurring position 1 for the pyrimidine bases and position 9 for the purine bases according to the standard numbering convention.

In one embodiment, the prodrug strategy involves masking the reactive groups, e.g. the charged —OH and phosphate groups in vivo, to permit passage across cell membranes.

In one embodiment, the present invention provides prodrugs of Formula I:

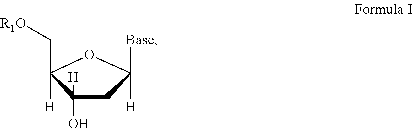

wherein Base refers to an optionally substituted heterocyclic base or an optionally substituted heterocyclic base with a protected amino group;

R¹ is selected from the group consisting of optionally substituted acyl, optionally substituted O-linked amino acid,

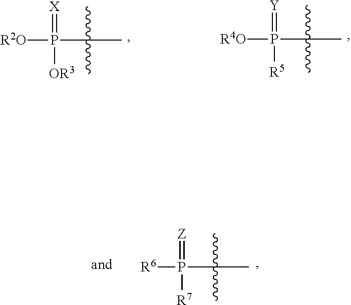

X, Y and Z are each independently selected from O and S;

R², R³ and R⁴ are each independently selected from hydrogen, optionally substituted C₁₋₂₄ alkyl, optionally substituted C₂₋₂₄ alkenyl, optionally substituted C₂₋₂₄ alkynyl, optionally substituted C₃₋₆ cycloalkyl, optionally substituted C₃₋₆ cycloalkenyl, optionally substituted aryl, optionally substituted heteroaryl, optionally substituted aryl(C₁₋₆) alkyl,

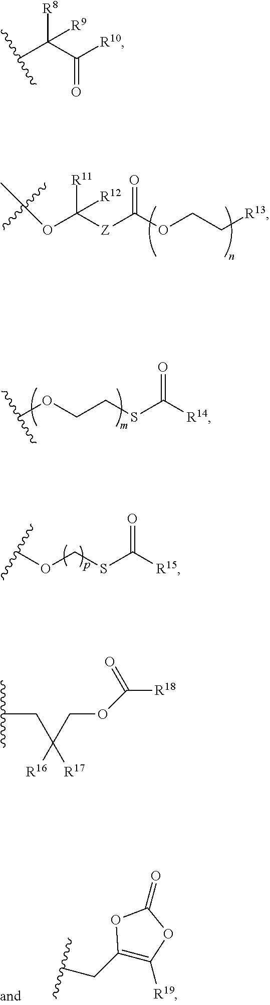

or R² and R³ can be taken together to form a cyclic moiety;

R⁵, R⁶ and R⁷ are each independently selected from optionally substituted C₁₋₂₄ alkyl, optionally substituted C₂₋₂₄ alkenyl, optionally substituted C₂₋₂₄ alkynyl, optionally substituted C₃₋₆ cycloalkyl, optionally substituted C₃₋₆ cycloalkenyl, NR²⁰R²¹, optionally substituted N-linked amino acid, optionally substituted N-linked amino acid ester;

R⁸, R⁹, R¹¹ and R¹² are each independently selected from hydrogen, optionally substituted C1-24 alkyl and optionally substituted aryl;

R¹⁰ and R¹³ are each independently selected from hydrogen, optionally substituted C1-24 alkyl and optionally substituted aryl, an optionally substituted —O-C1-24 alkyl, an optionally substituted —O-aryl, an optionally substituted —O-heteroaryl, an optionally substituted —O-monocyclic hetercyclyl;

R¹⁴, R¹⁵ and R¹⁹ are each independently selected from hydrogen, an optionally substituted C₁₋₂₄ alkyl and an optionally substituted aryl;

R¹⁶ and R¹⁷ are each independently selected from —CN, optionally substituted C₂₋₈ organylcarbonyl, C₂₋₈ alkoxycarbonyl and C₂₋₈ organylaminocarbonyl;

R¹⁸ is selected from hydrogen, optionally substituted C₁₋₂₄ alkyl, optionally substituted C₂₋₂₄ alkenyl, optionally substituted C₂₋₂₄ alkynyl; optionally substituted C₃₋₆ cycloalkyl and optionally substituted C₃₋₆ cycloakenyl;

R²⁰ and R²¹ are each independently selected from hydrogen, optionally substituted C₁₋₂₄ alkyl, optionally substituted C₂₋₂₄ alkenyl, optionally substituted C₂₋₂₄ alkynyl; optionally substituted C₃₋₆ cycloalkyl and optionally substituted C₃₋₆ cycloakenyl; and

n, m and p are each independently selected from 0, 1, 2, or 3.

In a particular embodiment, the prodrug is a compound of Formula Ia:

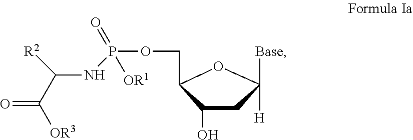

wherein each of R¹, R² and R³ are independently selected from hydrogen, optionally substituted C₁₋₂₄ alkyl, optionally substituted C₂₋₂₄ alkenyl, optionally substituted C₂₋₂₄ alkynyl, optionally substituted C₃₋₆ cycloalkyl, optionally substituted C₃₋₆ cycloalkenyl, optionally substituted aryl, optionally substituted heteroaryl and optionally substituted aryl(C₁₋₆) alkyl.

In a particular embodiment, R¹ is aryl, R² is C₁₋₂₄ alkyl and R³ is C₁₋₂₄ alkyl.

In another particular embodiment, R² is an amino acid side chain.

In a further particular embodiment, the prodrug is selected from one of the following compounds:

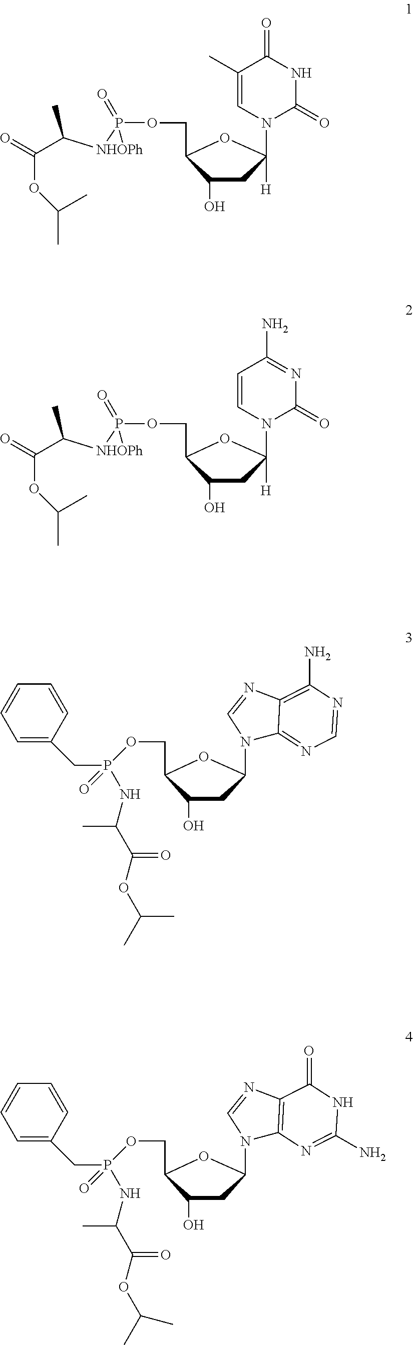

In another embodiment, the present invention provides prodrugs of Formula II:

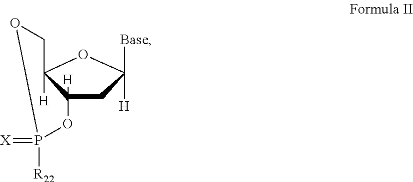

wherein Base refers to an optionally substituted heterocyclic base or an optionally substituted heterocyclic base with a protected amino group;

X is selected from S and O; and

R²² is selected from —O⁻, —OH, —O-alkyl, optionally substituted C₁₋₆ alkoxy,

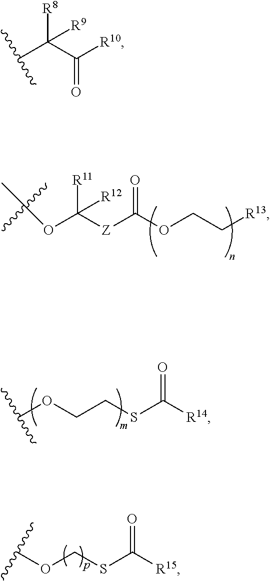

optionally substituted N-linked amino acid and optionally substituted N-linked amino acid ester;

wherein R⁸, R⁹, R¹⁰, R¹¹, R¹², R¹³, R¹⁴, R¹⁵, n, m and p are defined as above.

In a particular embodiment, the prodrug is a compound of Formula IIa:

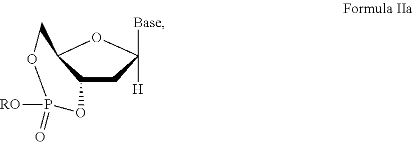

wherein R is C₁₋₄ alkyl.

In a still further particular embodiment, the prodrug is selected from one of the following compounds:

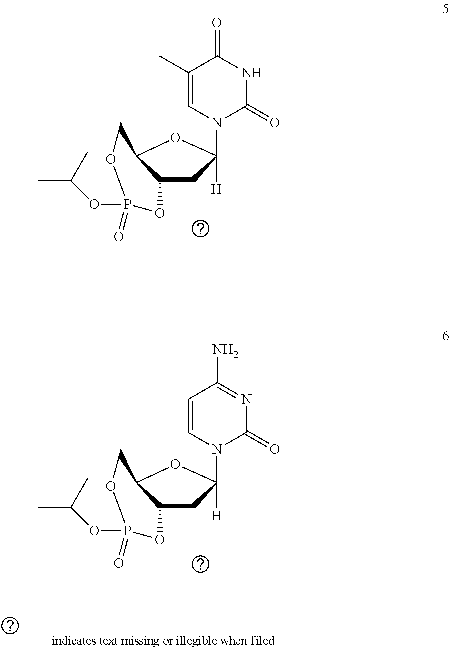

In a still further particular embodiment, the prodrug is selected from one of the following compounds:

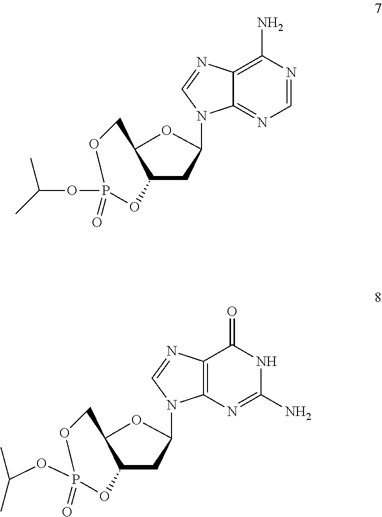

In still another embodiment, the present invention provides prodrugs of Formula III:

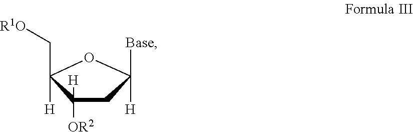

wherein Base refers to an optionally substituted heterocyclic base or an optionally substituted heterocyclic base with a protected amino group.

R¹ and R² are independent selected from hydrogen, phosphate (including mono-, di-, or tri-phosphate and the modified phosphates of Formula I); straight chained, branched or cyclic alkyl; optionally substituted acyl; CO-alkyl, CO-alkoxyalkyl; CO-aryloxyalkyl, CO-substituted aryl, sulfonate ester; alkylsulfonyl; arylsulfonyl; aralkylsulfonyl; a lipid; a phospholipid; an amino acid; a carbohydrate; a peptide and cholesterol.

Suitable bases can be unprotected or include a protected amino group. Examples of protecting groups that can be added to form the protected amino groups include without limitation, carbamate-protecting groups, such as 2-trimethylsilylethoxycarbonyl (Teoc), 1-methyl-1-(4-biphenylyl)ethoxycarbonyl (Bpoc), t-butoxycarbonyl (BOC), allyloxycarbonyl (Alloc), 9-fluorenylmethyloxycarbonyl (Fmoc), and benzyl-oxycarbonyl (Cbz); amide-protecting groups, such as formyl, acetyl, trihaloacetyl, benzoyl, and nitrophenylacetyl; sulfonamide-protecting groups, such as 2-nitrobenzenesulfonyl; and imine- and cyclic imide-protecting groups, such as phthalimido and dithiasuccinoyl.

In one embodiment, R² is an amino acid, i.e. the compound for Formula III is a 3′-amino acid ester, e.g. a compound of Formula IIIa:

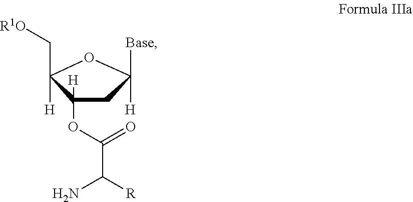

wherein R is the side chain of the amino acid.

In a particular embodiment the amino acid is valine (i.e. R is CH(CH₃)₂).

In a more particular embodiment, the prodrug is a compound of Formula IIIb:

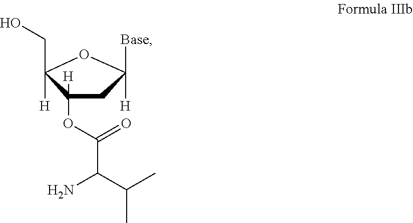

wherein Base refers to an optionally substituted heterocyclic base or an optionally substituted heterocyclic base with a protected amino group.

In a still more particular embodiment, the prodrug is selected from one of the following compounds:

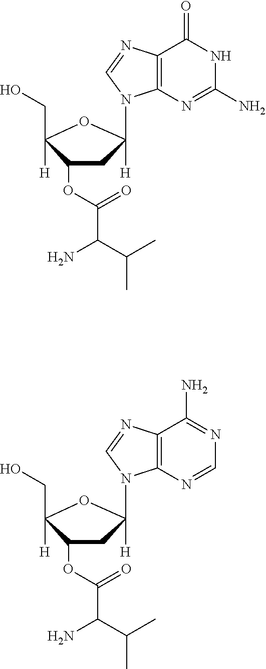

In a still further particular embodiment, the prodrug is selected from one of the following compounds:

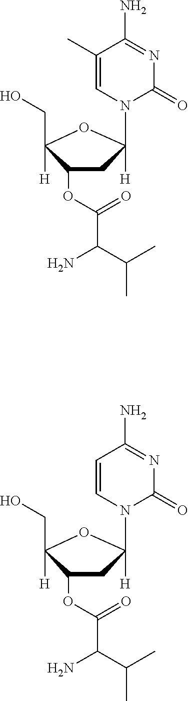

In a still further embodiment, the prodrug is a compound of Formula IIIc:

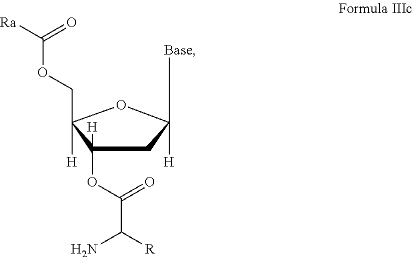

wherein Base refers to an optionally substituted heterocyclic base or an optionally substituted heterocyclic base with a protected amino group;

Ra is straight-chained, branched or cyclic alkyl and R is a side chain of an amino acid.

In a particular embodiment, Ra is selected from methyl, ethyl, n-propyl, isopropyl, n-butyl, sec-butyl, isobutyl and tent-butyl and R is CH(CH₃)₂).

In a still further particular embodiment, the prodrug is selected from one of the following compounds:

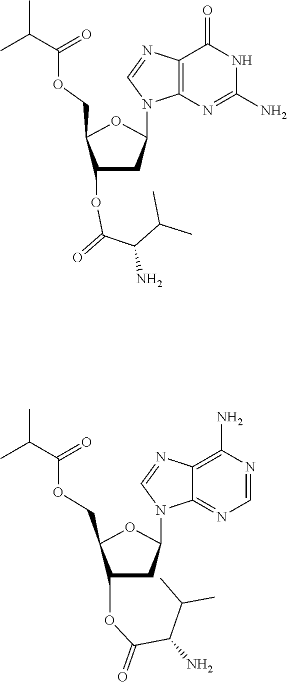 [SMILES: CC(C)C(=O)OC[C@H]1O[C@@H](n2cnc3c(=O)[nH]c(N)nc32)C[C@H]1OC(=O)[C@@H](N)C(C)C ; CC(C)C(=O)OC[C@H]1O[C@@H](n2cnc3c(N)ncnc32)C[C@H]1OC(=O)[C@@H](N)C(C)C]

In still another embodiment, the present invention provides prodrugs of Formula III wherein R¹ and R² are both substituted acyl groups wherein the acyl substitution can be C1-C6 alkyl.

In a particular embodiment, R¹ and R² are both isopropyl and the prodrug is one of the following compounds:

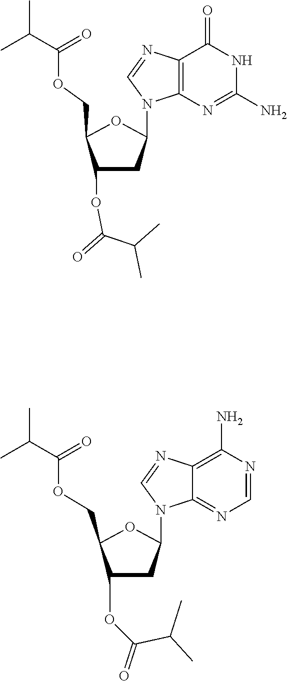 [SMILES: CC(C)C(=O)OC[C@H]1O[C@@H](n2cnc3c(=O)[nH]c(N)nc32)C[C@H]1OC(=O)C(C)C ; CC(C)C(=O)OC[C@H]1O[C@@H](n2cnc3c(N)ncnc32)C[C@H]1OC(=O)C(C)C]

### III. Methods of Use

The present invention provides a method of treating a disease or disorder characterized by unbalanced nucleotide pools in a subject in need thereof comprising administering to the subject a therapeutically effective amount of at least one prodrug described herein.

In one embodiment, prodrugs described hereinabove can be utilized in the present method.

The prodrug can be administered as such (i.e. alone) or in the form of a pharmaceutical composition. Pharmaceutical compositions comprising one of more prodrugs for administration may comprise a therapeutically effective amount of the prodrug and a pharmaceutically acceptable carrier. The phrase “pharmaceutically acceptable” refers to molecular entities and compositions that are physiologically tolerable and do not typically produce an allergic or similar untoward reaction, such as gastric upset, dizziness and the like, when administered to a human, and approved by a regulatory agency of the Federal or a state government or listed in the U.S. Pharmacopeia or other generally recognized pharmacopeia for use in animals, and more particularly in humans. “Carrier” refers to a diluent, adjuvant, excipient, or vehicle with which the therapeutic is administered. Such pharmaceutical carriers can be sterile liquids, such as saline solutions in water and oils, including those of petroleum, animal, vegetable, or synthetic origin, such as peanut oil, soybean oil, mineral oil, sesame oil, and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents.

In a particular embodiment, the disclosed method can be used to treat a mitochondrial DNA (mtDNA) depletion syndrome (MDS). Each nucleated cell contains several hundreds of mitochondria, which are unique organelles in being under dual genome control. The mitochondria contain their own DNA, the mtDNA, but most of mitochondrial proteins are encoded by nuclear genes, including all the proteins required for replication, transcription, and repair of mtDNA.

The maintenance of mtDNA requires proteins essential for mtDNA synthesis, for maintenance of the mitochondrial nucleotide pool, and for mediating mitochondrial fusion. The enzymes that synthesize mtDNA require a balanced supply of intramitochondrial nucleotides. These are supplied through mitochondrial nucleotide salvage pathways and the import of nucleotides from the cytosol via specific transporters. To function properly in mtDNA synthesis the quantities of these enzymes need to be balanced appropriately. The proteins known to be required for mtDNA synthesis are encoded by nuclear genes. When pathogenic variants disrupt the function of any one of the proteins encoded by these genes, mtDNA synthesis is impaired, resulting in either quantitative defects in mtDNA (mtDNA depletion) or qualitative defects in mtDNA (multiple mtDNA deletions). To date, pathogenic variants in more than 20 nuclear genes are known to be associated with mtDNA maintenance defects. One cause of mtDNA deletions is unbalanced nucleotide pools which can lead to replication errors. Although DNA polymerases discriminate between ribo- and deoxy-nucleotides, the system is not perfect and polymerases can misincorporate ribonucleotides, especially where pool imbalances exist.

In one embodiment, the mt DNA depletion syndrome is a nuclear DNA-based mtDNA depletion syndrome. In a particular embodiment, the nuclear DNA encodes a protein involved in nucleotide metabolism where an imbalance in free nucleotide concentrations leads to disturbances in mtDNA replication and mtDNA copy number decreases.

In a particular embodiment, the method of the present invention is useful in treating a disease or disorder caused by a deoxyribonucleoside triphosphate (dNTP) pool imbalance. dTNPs are the precursors used by DNA polymerases for replication and repair of nuclear and mitochondrial DNA in animal cells. The concentration of dNTPs depends on a balance between synthesis, consumption and degradation.

Accurate DNA synthesis requires adequate amounts of each dNTP and appropriately balanced dNTP pools. Total cellular pool sizes are in the range of 10-100 pmoles of each dNTP/million cells during S phase, with mitochondrial pools representing at most 10% of the total. In quiescent or differentiated cells pools are about 10-fold lower both in the cytosol and mitochondria. Contrary to what may be expected on the basis of the roughly equimolar abundance of the 4 nitrogen bases in DNA, the four dNTPs are present in the pools in different ratios, with pyrimidines often exceeding purines. Individual cell lines may exhibit different pool compositions even if they are derived from the same animal species. An increase in the concentration of one dNTP usually results in depletion of another dNTP.

The mtDNA depletion syndrome may involve a particular tissue or organ, e.g., the muscle, the liver, the brain and/or the GI tract. In a particular embodiment, the mtDNA depletion syndrome is a myopathic or hepato-cerebral syndrome.

Disorders characterized by unbalanced nucleotide pools include, but are not limited to nuclear genes that disrupt mitochondrial dNTP pools such as MPV17 and deoxyguanosine kinase (dGK), a defect parallel to TK2 deficiency due to autosomal recessive mutations in DGUOK resulting in deficiencies in dGMP and dAMP, with mtDNA depletion syndrome typically manifesting as early childhood-onset hepatocerebral disease (Mandel, et al. 2001). Disorders related to these genes can be treated with the methods herein. For example, for patients with dGK deficiencies, administration of prodrugs of dG and/or dA would logically be administered. In a particular embodiment of the present method, either dG or prodrugs of dG alone can be used to treat MPV17 or dGK deficiency disorders. Optionally dG or prodrugs of dG can be supplemented with smaller quantities of dA, dC or dT and salts and prodrugs thereof, intended to balance nucleoside pools, are useful in treating a patient with MPV17 or dGK deficiency disorders.

Molecular genetic testing using a panel of genes known to cause mtDNA depletion syndrome can be performed (Chanprasert, et al. 2012) and may be useful to identify patients with a disease or disorder characterized by unbalanced nucleotide pools. This testing can include a sequence analysis of the entire coding and exon/intron junction regions of a gene implicated by the diagnosis of a MDS for sequence variants and deletion/duplication. If compound heterozygous or homozygous deleterious mutations are identified in the sequence analysis, the diagnosis of deficiency is confirmed, and thus, the subject would benefit from the deoxynucleoside therapy. If sequence analysis does not identify two compound heterozygous or homozygous deleterious mutations, deletion/duplication analysis should be considered to determine and/or confirm a deficiency diagnosis.

Administration can be via any route including, but not limited to, oral, intrathecal, parental, mucosal and transdermal.

In one embodiment, administration is oral. Exemplary oral dosage forms include, but are not limited to, capsules, tablets, powders, granules, solutions, syrups, suspensions (in non-aqueous or aqueous liquids), or emulsions. Tablets or hard gelatin capsules may comprise lactose, starch or derivatives thereof, magnesium stearate, sodium saccharine, cellulose, magnesium carbonate, stearic acid or salts thereof. Soft gelatin capsules may comprise vegetable oils, waxes, fats, semi-solid, or liquid polyols. Solutions and syrups may comprise water, polyols, and sugars. The prodrugs described herein can be added to any form of liquid a patient would consume including but not limited to, milk, both cow's and human breast, infant formula, and water. The prodrug may be coated with or admixed with a material that delays disintegration and/or absorption in the gastrointestinal tract. Thus, the sustained release may be achieved over many hours.

In another embodiment, administration is intrathecal. Intrathecal administration involves injection of the drug into the spinal canal, more specifically the subarachnoid space such that it reaches the cerebrospinal fluid. This method is commonly used for spinal anesthesia, chemotherapy, and pain medication. Intrathecal administration can be performed by lumbar puncture (bolus injection) or by a port-catheter system (bolus or infusion). The catheter is most commonly inserted between the laminae of the lumbar vertebrae and the tip is threaded up the thecal space to the desired level (generally L3-L4). Intrathecal formulations most commonly use water, and saline as excipients but EDTA and lipids have been used as well.

In yet another embodiment, administration is parenteral, including intravenous. Pharmaceutical compositions adapted for parenteral administration include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerin, and vegetable oils. Compositions adapted for parental administration may be presented in unit- dose or multi-dose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: Water for Injection USP; aqueous vehicles such as Sodium Chloride Injection, Ringer's Injection, Dextrose Injection, Dextrose and Sodium Chloride Injection, and Lactated Ringer's Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate. Additionally, since some patients may be receiving enteral nutrition by the time treatment begins, the prodrug(s) can be administered through a gastronomy feeding tube or other enteral nutrition means.

Pharmaceutical compositions adapted for nasal and pulmonary administration may comprise solid carriers such as powders, which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

Pharmaceutical compositions adapted for rectal administration may be provided as suppositories or enemas. Pharmaceutical compositions adapted for vaginal administration may be provided as pessaries, tampons, creams, gels, pastes, foams or spray formulations.

Pharmaceutical compositions adapted for transdermal administration may be provided as discrete patches intended to remain in intimate contact with the epidermis of the recipient over a prolonged period of time.

The amount of the at least one prodrug or composition comprising the same administered can be from about 15 mg/kg/day to about 500 mg/kg/day. A further preferred dose ranges from about 50 mg/kg/day to about 400 mg/kg day. A further preferred dose ranges from about 100 mg/kg/day to about 350 mg/kg/day, such as, for example, from about 150 mg/kg/day to about 300 mg/kg/day, from about 200 mg/kg/day to about 350 mg/kg/day or from about 250 mg/kg/day to about 350 mg/kg/day.

In one embodiment, the dose is from about 20% equimolar to about 100% equimolar to the canonical nucleoside (dT, dC, dG, dA), e.g. from about 20% to about 80%, from about 20% to about 50%, from about 50% to about 80% or about 50% to about 100%. In a particular embodiment, the dose is about 20% equimolar to the canonical nucleoside. In another particular embodiment, the dose is about 50% equimolar to the canonical nucleoside. In still another embodiment, the dose is about 100% equimolar to the canonical nucleoside.

Administration of the at least one prodrug or composition comprising the same can be once a day, twice a day, three times a day, four times a day, five times a day, up to six times a day, preferably at regular intervals. Doses can also be lowered if being administered intravenously or intrathecally. Preferred dose ranges for such administration are from about 5 mg/kg/day to about 200 mg/kg/day.

In embodiments wherein the composition comprises more than one prodrug, the ratio of the prodrugs can vary. For example, if a dG prodrug is to be administered with a second deoxynucleoside (dN), the ratio of dG/dN can be 95/5, 90/10, 85/15, 80/20, 75/25, 70/30, 65/35, 60/40, 55/45 or 50/50.

In one embodiment, the method further comprises monitoring the subject for improvement of their condition prior to increasing the dosage. A subject's response to the therapeutic administration can be monitored by observing a subject's muscle strength and control, and mobility as well as changes in height and weight. If one or more of these parameters increase after the administration, the treatment can be continued. If one or more of these parameters stays the same or decreases, the dosage can be increased.

In another embodiment, the method further comprises monitoring the subject for adverse reactions prior to decreasing the dosage. Exemplary adverse effects include, but are not limited to, diarrhea, abdominal bloating and other gastrointestinal manifestations.

The prodrugs of the present invention can also be co-administered with other agents. Such agents would include therapeutic agents for treating the symptoms of the particular form of MDS. In particular, for MPV17 or dGK deficiency diseases, other agents, including an inhibitor of ubiquitous nucleoside catabolic enzymes, including but not limited to enzyme inhibitors such as tetrahydrouridine (inhibitor of cytidine deaminase) and immucillin H (inhibitor of purine nucleoside phosphorylase) and tipiracil (inhibitor of thymidine phosphorylase). Such inhibitors are known and used in the treatment of some cancers.

EXAMPLES

### Example 1

### Prodrugs Testing Identification Numbers

| | | Structures |
|---|---|---|
| | | R chosen from Cytosine, Thymine, Guanine, or Adenine- |
| STRUCTURE | Short-hand | having the same connection points and stereochemistry as naturally occurring |
| Code # | description | deoxynucleosides |
| MT101C MT101T MT101G MT101A | 3′ Val dC 3′ Val dT 3′ Val dG 3′ Val dA | 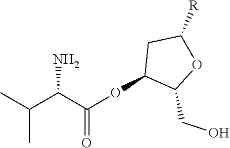 |
| MT102C MT102T MT102G MT102A | Cyclic phosphate prodrug of dC Cyclic phosphate prodrug of dT Cyclic phosphate prodrug of dG Cyclic phosphate prodrug of dA | 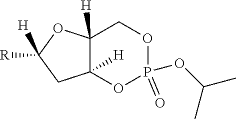 |
| MT103C MT103T MT103G MT103A | Phosphoramidate prodrug of dC Phosphoramidate prodrug of dT Phosphoramidate prodrug of dG Phosphoramidate prodrug of dA | 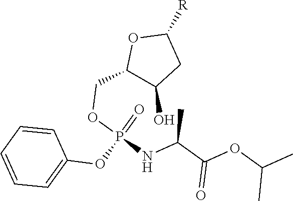 |
| MT102C MT102T MT102G MT102A | Cyclic phosphate prodrug of dC Cyclic phosphate prodrug of dT Cyclic phosphate prodrug of dG Cyclic phosphate prodrug of dA | 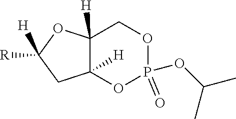 |
| MT104C MT104T MT104G MT104A | 3′Val, 5′isobutryl dC 3′Val, 5′isobutryl dT 3′Val, 5′isobutryl dG 3′Val, 5′isobutryl dA | 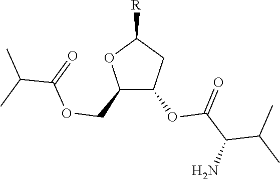 |
| MT105C MT105T MT105G MT105A | 3′5′-di-isobutyryl dC 3′5′-di-isobutyryl dT 3′5′-di-isobutyryl dG 3′5′-di-isobutyryl dA | 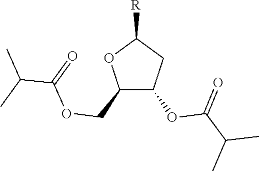 |

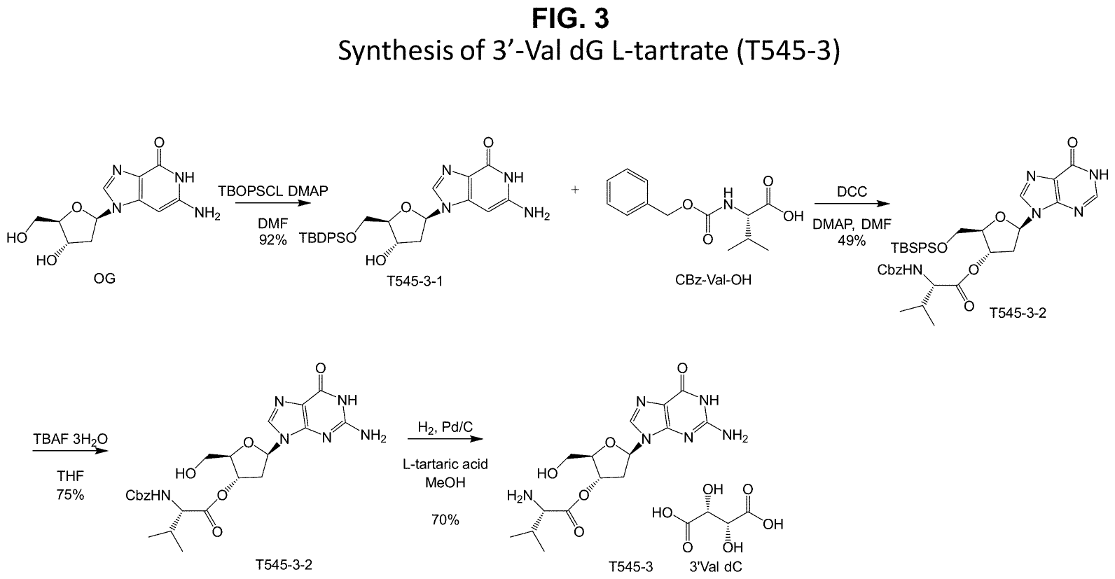

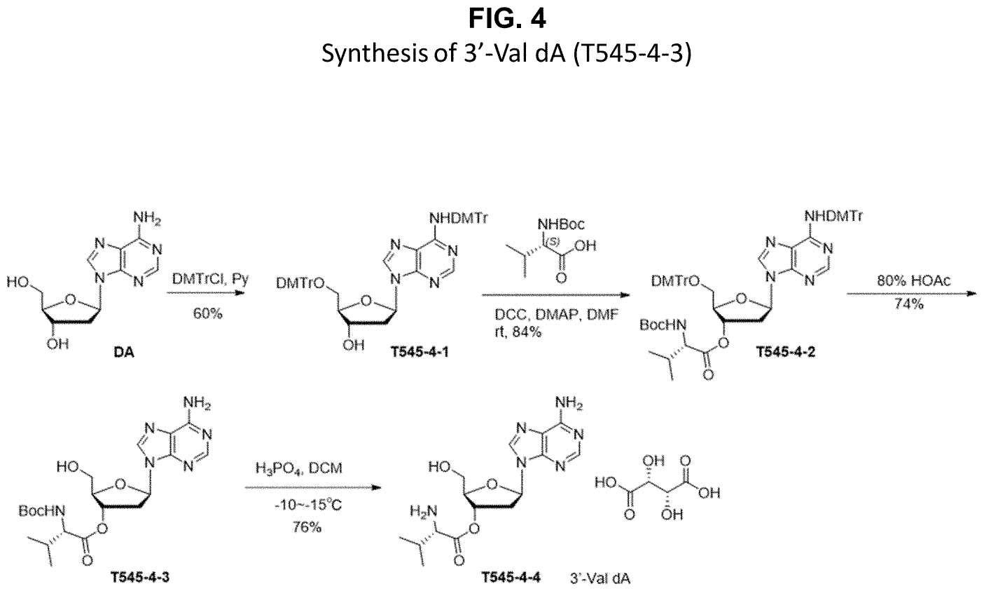

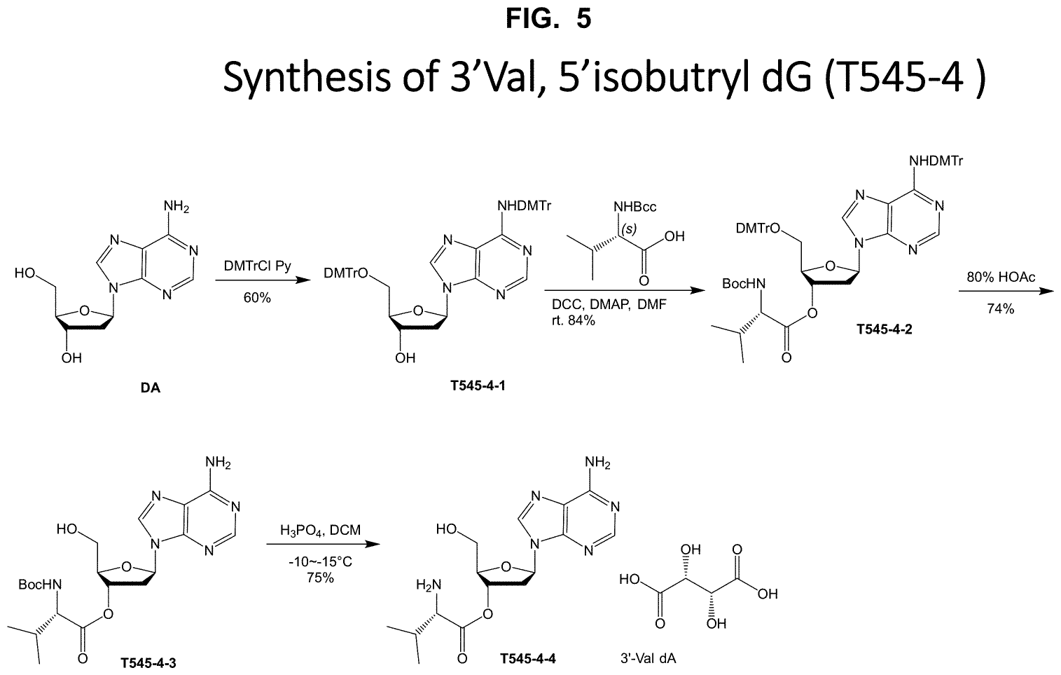

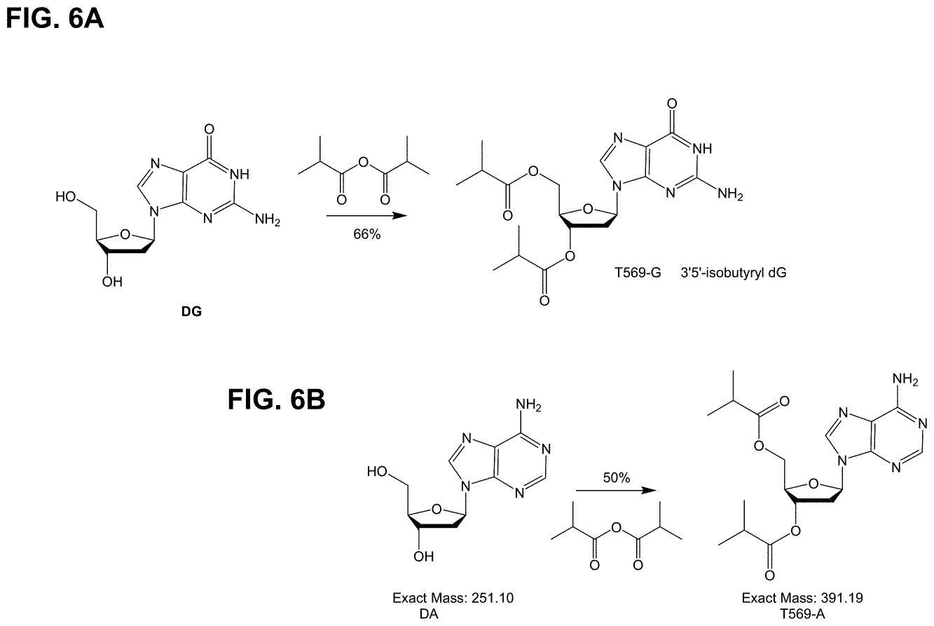

Synthesis of pro-drugs was accomplished using protection/deprotection strategies known in the art for nucleoside and derivatives of nucleosides. Syntheses were performed with commercially available, chemical grade nucleoside starting materials, such starting materials are available from numerous suppliers such as, for example, Hongene Biotech Corporation, Shanghai, China. General schemes for the syntheses of compounds in Examples 1A to 1D are depicted in FIGS. 3, 4, 5, 6A, and 6B.

### 1A. Synthetic Description for MT101-G

Step 1: Starting with deoxyguanosine, the 5′-hydroxyl group of deoxyguanosine was selectively protected by addition of a TBDPS (t-butyldiphenylsilyl) group under reaction conditions of t-butyldiphenylsilyl chloride (TBDPSCl)/dimethylaminopyridine (DMAP)in dimethylformamide (DMF) at room temperature (rt).

Step 2: The esterification of the 3′hydroxyl group was accomplished with (carbobenzoxy) Cbz-L-Valine and dicyclohexylcarbodiimide (DCC) in CH₂Cl₂ (DCM) to give the protected intermediate T545-3-2, which was purified by silica column chromatography for a total yield of 49%.

Step 3: The TBDPS group was then removed using n-Bu₄NF (TBAF) in tetrahydrofuran (THF) at rt to give T545-3-3 in 75% yield after column purification.

Step 4: Hydrogenation using hydrogen gas in solution with carbon on palladium catalyst in the presence of L-tartaric acid gave MT101-G as the L-tartaric salt.

### 1B. Synthetic Description for MT101-A

Step 1: Starting with deoxyadenosine, the 5′-hydroxyl group was selectively protected by a 4,4′-dimethoxytrityl (DMTr) group in pyridine at 0-10° C. to give T545-4-1 with a yield of 60% after column purification.

Step 2: The esterification of 3′- hydroxyl group was accomplished as in Ex. 1A by Cbz-L-Valine and DCC in DCM at rt to give T545-4-2, which was purified by silica column chromatography to provide a total yield of 84%.

Step 3: The removal of DMTr group was realized with aq. 80% AcOH at rt. The product was purified by silica column chromatograph in 74% yield.

Step 4: The condition of H₃PO₄/DCM was employed for the de-Boc protection, then charging of L-tartaric acid was used to give MT101-A as the L-tartrate salt in 76% yield by crystallization.

### 1C. Synthetic Description for MT105-G

3′5′-isobutyryl dG was obtained by direct treatment of dG with isobutyric anhydride and obtained in 66% yield after column purification.

### 1D. Synthetic Description for MT105-A

3′5′-isobutyryl dA was obtained by direct treatment of dA with isobutyric anhydride and obtained in 50% yield after column purification.

### Example 2. Deoxynucleoside Supplementation Studies in MPV17 Mice

2A. 2 litters from MPV17^{-/-} mouse (KO×KO) breeding pairs were used in the study. The experimental groups were set up as follows:

1.—KO untreated (n=1)
2.—KO treated with 109.35 mg/kg MT102C+104.2 mg/kg MT102G (n=3)
3.—KO treated with 163.87 mg/kg MT103C+150.56 mg/kg MT103G (n=3)
4.—KO treated with 75 mg/kg dC and 75 mg/kg dG (purchased from Sigma) (n=3)
5.—KO treated with 144.9 mg/kg MT101G (n=2)

Treatment was started at day 7 and terminated at day 30. The treatment was performed by delivery into the oral cavity throughout the study. No altered health or mortality was observed in any of the experimental groups. At day 30, animals were sacrificed and tissues collected. Liver, muscle, kidney, gut, heart and brain were flash frozen in liquid nitrogen. Blood was collected by heart puncture and plasma fractions stored at −80° C.

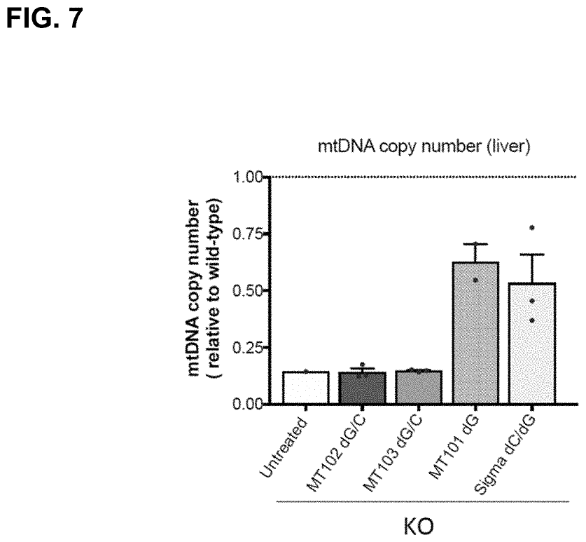

Results of qPCR measurements of mtDNA copy number in the liver tissue of treated and untreated mice are shown FIG. 7.

2B. In order to compare results, WT (n=5), KO untreated (n=4) and KO treated with 20% MT101 (n=4) and 20% MT104 (n=4) from previous experiments were also included in the analysis.

2C. Three other litters from KO×KO breeding pairs were used in a further study of nucleoside prodrug supplementation:

### 1.—Litter 2 (L2): Born on Feb. 9, 2020

### 2.—Litter 3 (L3): Born on May 9, 2020

### 3.—Litter 4 (L4): Born on Aug. 9, 2020

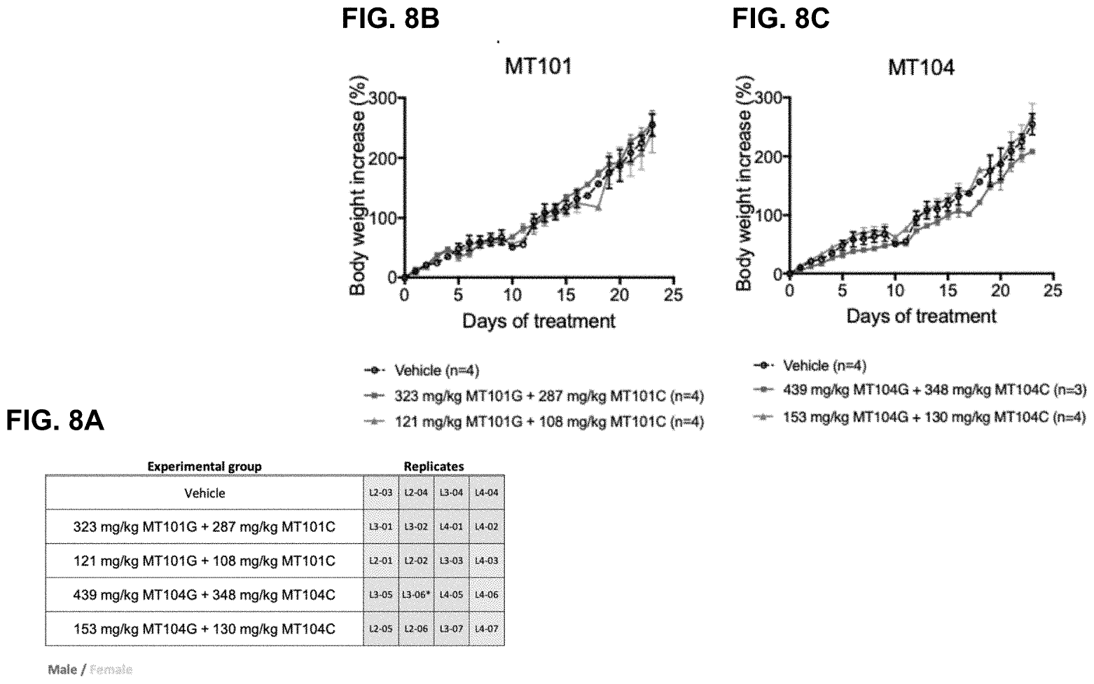

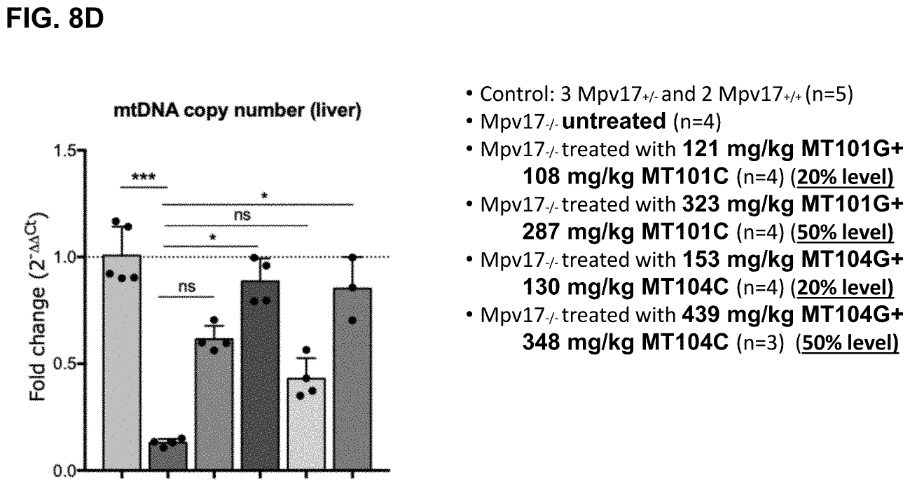

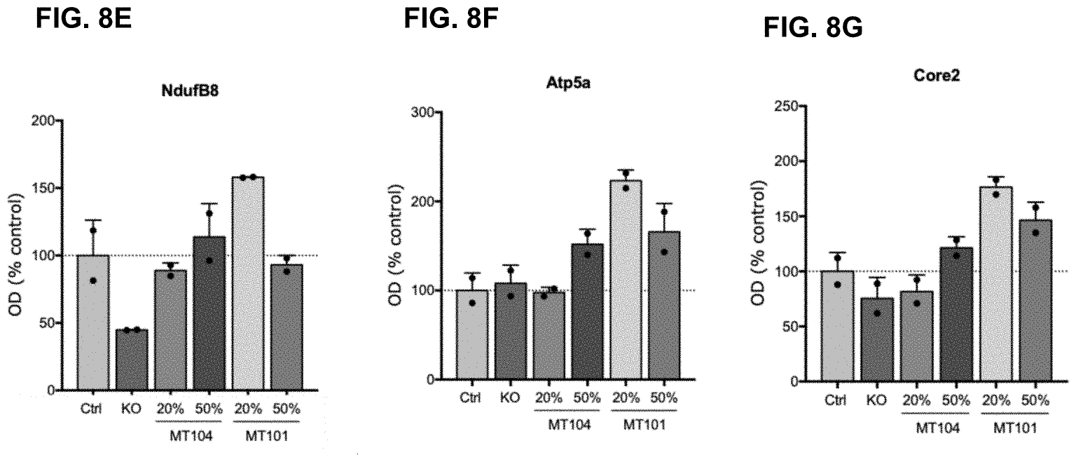

The experimental groups were set up as shown in FIG. 8A which shows dosing amounts and replicates and sexes of test animals from the different litters set out above.

The treatment started at day 7 and terminated at day 30. The treatment was performed by delivery into the oral cavity throughout the study. No distress was noted; rather, after a few days, the animals became accustomed to the technique.

Body weight of each animal was monitored daily to adjust the dosage and to detect any possible detrimental effect of the compounds. No altered health or mortality was observed in any of the experimental groups and weight gains of treated pups were similar to untreated as shown for prodrugs MT101 and MT104 in FIGS. 8B and 8C, respectively.

At day 30, animals were sacrificed and tissues collected. Liver, muscle, kidney, gut, heart and brain were flash frozen in liquid nitrogen. Blood was collected by heart puncture and plasma fractions stored at −80° C.

Liver and muscle, which manifest marked mtDNA depletion at day 6, are being analyzed. The effect on mtDNA copy number for liver is shown in FIG. 8D. The effects of the nucleoside supplementation on mitochondrial OXPHOS proteins are shown in FIGS. 8E, 8F, and 8G.

Using the same procedure in Example 2A, the experimental groups were set up to receive differing combinations of supplementation as follows:

1. KO untreated (n=1)
2. KO treated with 20% MT101G (n=2)
3. KO treated with 20% MT101C (n=4)
4. KO treated with 20% MT105G (n=4)
5. KO treated with 75 mg/kg dG (Sigma, D7145) (n=4)
6. KO treated with 75 mg/kg dC (Sigma, D3897) (n=2)
20% represents the molar equivalent of 75 mg/kg dC or 75 mg/kg dG.

mtDNA copy number in liver was analyzed by RT-qPCR. In order to compare results, WT (n=5), KO untreated (n=4) and KO treated with 20% MT101G (n=2) from previous experiments were also included in the analysis.

### Example 3: dGK Deficient Patient Derived Fibroblasts

Prodrugs were tested in dGK deficient patient derived fibroblasts to determine their effect on mtDNA copy number. An advantage of working with dGK-deficient fibroblasts is that they spontaneously undergo mtDNA depletion after quiescence is induced without the addition of DNA damaging agents. It has been previously demonstrated that supplementing the cell culture medium with dGuo (50 μM) was sufficient to prevent this depletion. Cells were grown up to confluence. Quiescence was induced by reducing FBS in the medium to 0.1%. Three days later (day 0), we supplemented cell culture medium with 50 μM of dGuo (deoxyguanosine) or prodrug of the present invention. Cells were maintained in the same conditions up to 18 days with regular addition of fresh medium. mtDNA copy number was evaluated at different timepoints throughout the experiment (day 4, 9 and 18). Results were expressed as the mean+SD of experimental duplicates, and plotted as the mtDNA/nDNA ratios respect to the average values obtained for three untreated healthy controls cultured in parallel.

Equimolar concentrations of dGuo and the other dG prodrugs available (50 μM) were tested in parallel under similar conditions. Results showed that a 50/50 mixture of 7 and 8 at 50 μM prevented mtDNA depletion

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method for treating a mitochondrial depletion disease or disorder chosen from MPV17 and dGK deficiency in a subject in need thereof comprising administering to the subject a therapeutically effective amount of at least one prodrug of Formula I: 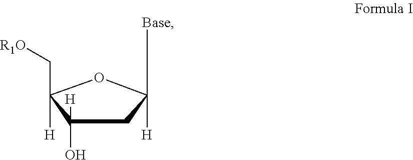
wherein Base refers to guanine or guanine with a protected amino group;
R¹ is selected from the group consisting of optionally substituted acyl, optionally substituted O-linked amino acid, 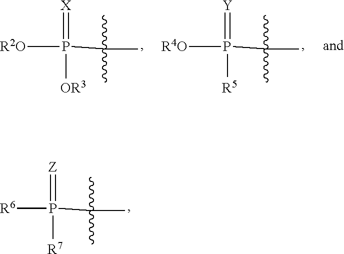
X, Y and Z are each independently selected from O and S;
R², R³ and R⁴ are each independently selected from hydrogen, optionally substituted C₁₋₂₄ alkyl, optionally substituted C₂₋₂₄ alkenyl, optionally substituted C₂₋₂₄ alkynyl, optionally substituted C₃₋₆ cycloalkyl, optionally substituted C₃₋₆ cycloalkenyl, optionally substituted aryl, optionally substituted heteroaryl, optionally substituted aryl(C₁₋₆) alkyl, 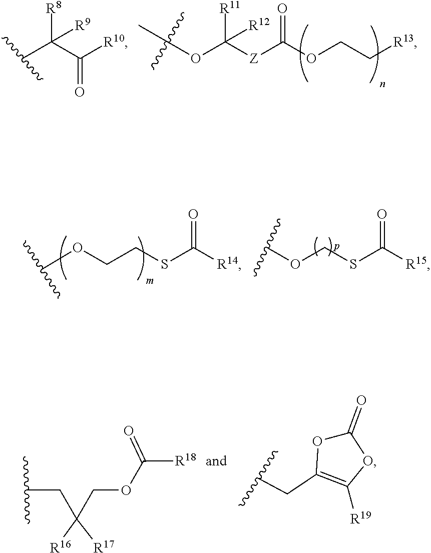
or R² and R³ can be taken together to form a cyclic moiety;
R⁵, R⁶ and R⁷ are each independently selected from optionally substituted C₁₋₂₄ alkyl, optionally substituted C₂₋₂₄ alkenyl, optionally substituted C₂₋₂₄ alkynyl, optionally substituted C₃₋₆ cycloalkyl, optionally substituted C₃₋₆ cycloalkenyl, NR²⁰R²¹, optionally substituted N-linked amino acid, optionally substituted N-linked amino acid ester;
R⁸, R⁹, R¹¹ and R¹² are each independently selected from hydrogen, optionally substituted C₁₋₂₄ alkyl and optionally substituted aryl;
R¹⁰ and R¹¹ are each independently selected from hydrogen, optionally substituted C₁₋₂₄ alkyl and optionally substituted aryl, an optionally substituted O-C₁₋₂₄ alkyl, an optionally substituted —O-aryl, an optionally substituted —O-heteroaryl, an optionally substituted —O-monocyclic hetercyclyl;
R¹⁴, R¹⁵ and R¹⁹ are each independently selected from hydrogen, an optionally substituted C₁₋₂₄ alkyl and an optionally substituted aryl;
R¹⁶ and R¹⁷ are each independently selected from —CN, optionally substituted C₂₋₈ organylcarbonyl, C₂₋₈ alkoxycarbonyl and C₂₋₈ organylaminocarbonyl;
R¹⁸ is selected from hydrogen, optionally substituted C₁₋₂₄ alkyl, optionally substituted C₂₋₂₄ alkenyl, optionally substituted C₂₋₂₄ alkynyl; optionally substituted C₃₋₆ cycloalkyl and optionally substituted C₃₋₆ cycloakenyl;
R²⁰ and R²¹ are each independently selected from hydrogen, optionally substituted C₁₋₂₄ alkyl, optionally substituted C₂₋₂₄ alkenyl, optionally substituted C₂₋₂₄ alkynyl; optionally substituted C₃₋₆ cycloalkyl and optionally substituted C₃₋₆ cycloakenyl; and
n, m and p are each independently selected from 0, 1, 2, or 3.

2. The method of claim 1, wherein the prodrug is a compound of Formula Ia: 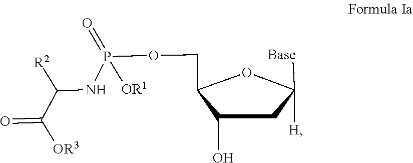
wherein each of R¹, R² and R³ are independently selected from hydrogen, optionally substituted C₁₋₂₄ alkyl, optionally substituted C₂₋₂₄ alkenyl, optionally substituted C₂₋₂₄ alkynyl, optionally substituted C₃₋₆ cycloalkyl, optionally substituted C₃₋₆ cycloalkenyl, optionally substituted aryl, optionally substituted heteroaryl and optionally substituted aryl(C₁₋₆) alkyl.

3. The method of claim 2, wherein R¹ is aryl, R² is C₁₋₂₄ alkyl and R³ is C₁₋₂₄ alkyl.

4. A method for treating a mitochondrial depletion disease or disorder chosen from MPV17 and dGK deficiency in a subject in need thereof comprising administering to the subject a therapeutically effective amount of at least one prodrug of Formula II 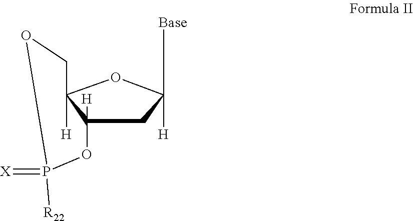
wherein Base refers to guanine or guanine with a protected amino group;
X is selected from S and O; and
R²² is selected from —O⁻, —OH, —O-alkyl, optionally substituted C₁₋₆ alkoxy, 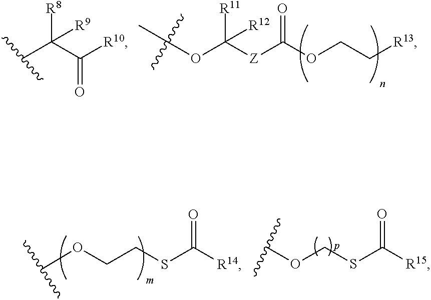
optionally substituted N-linked amino acid and optionally substituted N-linked amino acid ester;
wherein R⁸, R⁹, R¹¹ and R¹² are each independently selected from hydrogen, optionally substituted C₁₋₂₄ alkyl and optionally substituted aryl;
R¹⁰ and R¹³ are each independently selected from hydrogen, optionally substituted C₁₋₂₄ alkyl and optionally substituted aryl, an optionally substituted —O-C₁₋₂₄ alkyl, an optionally substituted —O-aryl, an optionally substituted —O-heteroaryl, an optionally substituted —O-monocyclic hetercyclyl; and
R¹⁴ and R¹⁵ are each independently selected from hydrogen, an optionally substituted C₁₋₂₄ alkyl and an optionally substituted aryl; and
n, m and p are each independently selected from 0, 1, 2, or 3.

5. The method of claim 4, wherein the prodrug is a compound of Formula IIa: 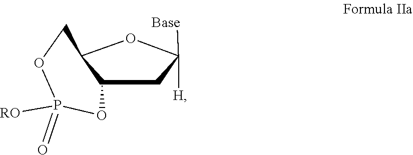
wherein R is C₁₋₄ alkyl.

6. A method for treating a mitochondrial depletion disease or disorder chosen from MPV17 and dGK deficiency in a subject in need thereof comprising administering to the subject a therapeutically effective amount of at least one prodrug of Formula III: 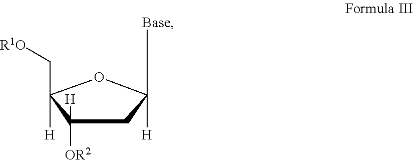
wherein Base refers to an optionally substituted heterocyclic base or an optionally substituted heterocyclic base with a protected amino group;
R¹ and R² are independently selected from hydrogen, phosphate; straight chained, branched or cyclic alkyl; acyl; CO-alkyl, CO-alkoxyalkyl; CO-aryloxyalkyl, CO-substituted aryl, sulfonate ester; alkylsulfonyl; arylsulfonyl; aralkylsulfonyl; a lipid; a phospholipid; an amino acid; a carbohydrate; a peptide and cholesterol.

7. The method of claim 6, wherein the prodrug is a compound of Formula IIIa: 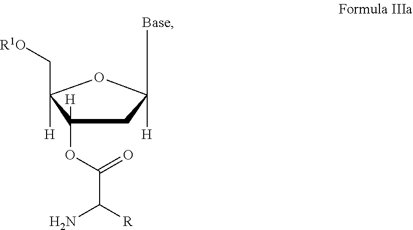
wherein R is the side chain of an amino acid.

8. The method of claim 6, wherein the prodrug is a compound of Formula IIIb: 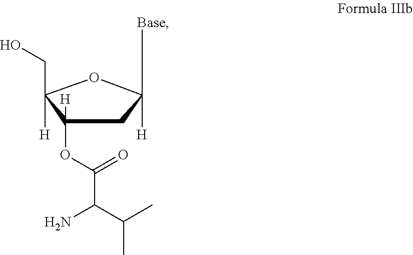
wherein Base refers to an optionally substituted heterocyclic base or an optionally substituted heterocyclic base with a protected amino group.

9. The method of claim 1 wherein the at least one prodrug is:

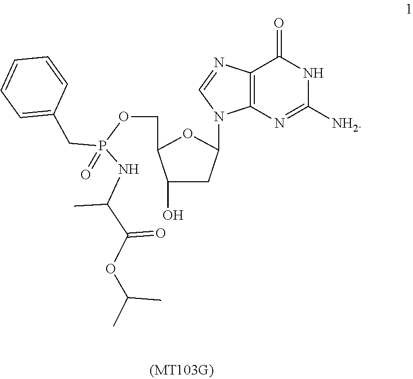 [SMILES: CC(C)OC(=O)C(C)NP(=O)(Cc1ccccc1)OCC1OC(n2cnc3c(=O)[nH]c(N)nc32)CC1O]

10. The method of claim 6, wherein a second prodrug is administered, and wherein the Base of the second prodrug is cytosine.

11. The method of claim 10, wherein the weight ratio of the at least one prodrug of Formula III to the second prodrug is 95/5, 90/10, 85/15, 80/20, 75/25, 70/30, 65/35, 60/40, 55/45 or 50/50.

12. The method of claim 6, wherein the prodrug is administered in the form of a pharmaceutical composition.

13. The method of claim 6, wherein the method of administration is oral.

14. The method of claim 6, wherein the dose administered is from about 200 mg/kg/day to about 1,000 mg/kg/day.

15. The method of claim 6, wherein the prodrug or composition comprising the same is administered at least once per day.

16. The method of claim 6, wherein Base refers to guanine.

17. The method of claim 6, wherein Base refers to guanine with a protected amino group.

18. The method of claim 4, wherein the at least one prodrug is:

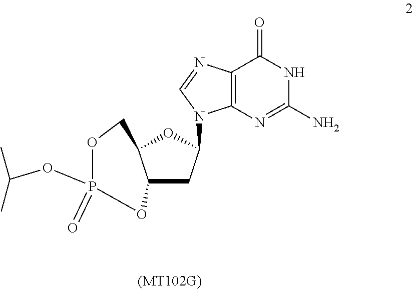

19. The method of claim 6, wherein the at least one prodrug is selected from the following: [chemical structure]
